(19) United States
(12) Patent Application Publication
(10) Pub. No.: US 2022/0042117 A1
(43) Pub. Date: Feb. 10, 2022

(54) COMPOSITIONS AND METHODS FOR THE SIMULTANEOUS DETECTION OF INFLUENZA A, INFLUENZA B, AND SEVERE ACUTE RESPIRATORY SYNDROME CORONAVIRUS 2 (SARS-CoV-2)

(71) Applicant: Roche Molecular Systems, Inc., Pleasanton, CA (US)

(72) Inventors: Renjian Huang, Belmont, MA (US); Jingtao Sun, San Ramon, CA (US)

(21) Appl. No.: 17/393,742

(22) Filed: Aug. 4, 2021

Related U.S. Application Data
(60) Provisional application No. 63/062,105, filed on Aug. 6, 2020.

(51) Int. Cl. C12Q 1/70

(52) U.S. Cl. CPC C12Q 1/701

(57) ABSTRACT

Methods for the rapid detection of the presence or absence of SARS-CoV-2 in biological or non-biological samples are described. These methods are adapted to be performed rapidly in a point-of-care setting. The methods can include performing an amplifying step, a hybridizing step, and a detecting step. Specifically, primers and probes targeting SARS-CoV-2 are provided that are designed for the detection of this target. Additionally, kits and reaction vessels containing primers and probes targeting SARS-CoV-2 are provided. Additionally, methods, kits and reaction vessels for the simultaneous rapid detection of the presence or absence of SARS-CoV-2, influenza A, and influenza B in biological or non-biological samples are described.

### CROSS-REFERENCE TO RELATED APPLICATIONS

This application claims priority to U.S. Provisional Patent Application No. 63/062,105, filed 6 Aug. 2020, the content of which is incorporated herein by reference in its entirety.

### REFERENCE TO SEQUENCE LISTING

The official copy of the Sequence Listing is submitted concurrently with the specification as an ASCII-formatted text file with a file name of “36305US_ST25”, a creation date of Aug. 5, 2020, and a size of 7,241 bytes. The Sequence Listing filed herewith is part of the specification and is incorporated in its entirety by reference herein.

### FIELD OF THE INVENTION

The present disclosure relates to the field of viral diagnostics, and more particularly to detection of the presence or absence of Severe Acute Respiratory Syndrome Coronavirus 2 (“SARS-CoV-2”) in samples. The present disclosure also relates to the simultaneous detection of the presence or absence of SARS-CoV-2, influenza A, and influenza B in samples.

### BACKGROUND OF THE INVENTION

Viruses of the family Coronaviridae possess a single stranded, positive-sense RNA genome ranging from 26 to 32 kilobases in length. Coronaviruses have been identified in several avian hosts, as well as in various mammals, including camels, bats, masked palm civets, mice, dogs, and cats. Novel mammalian coronaviruses are now regularly identified. For example, an HKU2-related coronavirus of bat origin was responsible for a fatal acute diarrhea syndrome in pigs in 2018.

Among the several coronaviruses that are pathogenic to humans, most are associated with mild clinical symptoms, with a few notable exceptions: severe acute respiratory syndrome (SARS) coronavirus (SARS-CoV), a novel betacoronavirus that emerged in Guangdong, southern China, in November, 2002, and resulted in more than 8000 human infections and 774 deaths in 37 countries during 2002-03; and Middle East respiratory syndrome (MERS) coronavirus (MERS-CoV), which was first detected in Saudi Arabia in 2012 and was responsible for 2494 laboratory-confirmed cases of infection and 858 fatalities since September, 2012, including 38 deaths following a single introduction into South Korea.

In late 2019, several patients with viral pneumonia were found in Wuhan, in the Hubei province of China. A novel, human-infecting coronavirus, initially named 2019 novel coronavirus (2019-nCoV), was identified by next-generation sequencing. This novel coronavirus is classified under the family Coronavirus, genus Betacoronavirus and subgenus Sarbecovirus, and is described in “Genomic characterization and epidemiology of 2019 novel coronavirus: implications for virus origins and receptor binding” by Lu, R. et al., Lancet, 2020, Vol. 395, p. 565-574. On Feb. 11, 2020, the World Health Organization (WHO) announced the formal name for the virus as Severe Acute Respiratory Syndrome Coronavirus 2 (SARS-CoV-2). As of Apr. 29, 2021, over 180 million confirmed cases and over 3.9 million fatalities had been reported internationally, and SARS-CoV-2 had been detected in nearly every country in the world, with the highest number in the United States (www.worldinfometers.com/coronavirus).

Influenza, or flu, is a highly contagious viral respiratory disease linked to viruses of the family Orthomyxoviridae. Flu infections can occur anytime, but are usually characterized by seasonal outbreaks during the winter months in each hemisphere. Symptoms vary widely in severity from patient to patient, but typically include one or more of cough, fever, runny or stuffy nose, sore throat, body aches, and fatigue. While flu can infect anyone, it is especially dangerous to the elderly and the very young, as well as those with diminished immune capacity and certain pre-existing conditions.

There are four known types of influenza virus, denoted influenza A through D. Humans can be infected by influenza A, B, and C, but no cases of influenza D infections of humans have been reported. The most common type infecting humans is influenza A, followed by influenza B. Influenza A is further divided into serotypes based on variations in two proteins, hemagglutinin (H) and neuraminidase (N), found on the outer surface of viral particles. Hemagglutinin variants H1-H3, and neuraminidase variants N1 and N2, form the most common serotypes that arise during seasonal outbreaks. In some years, influenza A outbreaks have had devastating worldwide impacts, resulting in flu pandemics. For example, the 1918 Spanish flu pandemic is estimated to have killed between 17 million and 50 million people. The Asian flu pandemic of 1957, and the Hong Kong flu pandemic of 1968, each killed a million or more people. The influenza A H1N1 serotype was responsible for the 1918 Spanish flu, while the H2N2 and H3N2 serotypes were the causative agents of the Asian flu and Hong Kong flu pandemics, respectively.

Influenza B and influenza C, while capable of infecting humans, are far less dangerous. Influenza B has a single serotype, and thus it is easier to establish and maintain population immunity against this virus. Influenza B is less prevalent than influenza A in humans, but disproportionately affects children and adolescents, and can lead to localized epidemics. Influenza C, while capable of infecting humans, is even less dangerous than influenza B, and patients exhibit only mild symptoms.

The SARS-CoV-2 genome is a positive sense single-stranded RNA molecule 29,903 bases in length (as shown in GenBank Accession No. MN908947) with the order of genes (5′ to 3′) as follows: replicase ORF1ab (21,291 bases with 16 predicted non-structural proteins that are essential for viral replication and viral assembly), spike (S gene, 3,822 bases coding for spike protein responsible for binding to cell receptor), ORF3ab (828 bases in length), envelope (E gene, 228 bases coding for envelope protein), membrane (M gene, 669 bases coding for membrane protein), nucleocapsid (N gene, 1260 bases coding for nucleocapsid protein that forms complexes with the genomic RNA). In addition, there are 265 bases of non-coding region at the 5′ terminal end and 229 bases of non-coding region at the 3′ terminal end.

The influenza A genome is a segmented negative sense single-stranded RNA molecule 13,588 bases in length (see www.ncbi.nlm.nih.gov/genomes/FLU/FLU.html). The genome is comprised of eight segments encoding between 10-14 genes, depending on the strain. From longest to shortest, the segments and the genes encoded thereon are: segment 1 (RNA polymerase subunit PB2); segment 2 (RNA polymerase subunit PB1 and PB1-F2 protein); segment 3 (RNA polymerase subunit PA and PA-X protein); segment 4 (hemagglutinin); segment 5 (nucleoprotein); segment 6 (neuraminidase); segment 7 (matrix protein M1 and matrix protein M2); and segment 8 (non-structural proteins NS1 and NEP). Hemagglutinin and neuraminidase are large proteins found on the exterior of the influenza virions. Hemagglutinin (HA) is responsible for binding of the influenza viral particles to the target cell and entry of the viral genome into the cell. Neuraminidase (NA) catalyzes release of virions from infected cells. There are 16 known subtypes of HA and nine of NA, but only H1, H2, and H3, and N1 and N2 are usually found in humans.

The influenza B genome, like the influenza A genome, is an eight-segmented negative sense single-stranded RNA molecule 14,548 bases in length. The genome of influenza B is very similar to that of influenza A, with a few exceptions. From longest to shortest, the segments and the genes encoded thereon are: segment 1 (RNA polymerase subunit PB2); segment 2 (RNA polymerase subunit PB1 protein); segment 3 (RNA polymerase subunit PA); segment 4 (hemagglutinin); segment 5 (nucleoprotein); segment 6 (neuraminidase and matrix protein NB); segment 7 (matrix protein M1 and membrane protein BM2); and segment 8 (non-structural proteins NS1 and NEP).

Rapid and accurate diagnosis and differentiation of SARS-CoV-2 and influenza infections is important in individuals suspected of a respiratory infection. The seasonality ranges of SARS-CoV-2 and influenza overlap and the clinical manifestations of the two diseases can be similar, ranging from asymptomatic or mild “influenza-like” illness (such as fever, cough, shortness of breath, or myalgia) in a majority of individuals to more severe and life-threatening disease. However, the two virus types differ in that SARS-CoV-2 patients can spread infection while presymptomatic, while influenza patients develop symptoms more quickly and do not shed virus while presymptomatic. As a result, rapid and accurate detection and differentiation of both SARS-CoV-2 and influenza can help to inform time-critical medical decision-making, facilitate infection control efforts, promote efficient resourcing, optimize use of targeted therapies and antimicrobials, and reduce ancillary testing or procedures. Thus, there is an ongoing need in the art for a rapid, reliable, specific, and sensitive method to detect SARS-CoV-2, as well as a method to detect and differentiate influenza A, influenza B, and SARS-CoV-2.

### SUMMARY OF THE INVENTION

The present disclosure provides methods for the rapid detection of the presence or absence of SARS-CoV-2 in a biological or non-biological sample, by qualitative or quantitative real-time reverse-transcription polymerase chain reaction (RT-PCR) in a single tube using a point of care (POC) device. Embodiments include methods of detection of SARS-CoV-2 comprising performing a reverse transcription step and at least one cycling step, which may include an amplifying step and a hybridizing step. Further embodiments include primers, probes, and kits that are designed for the detection of SARS-CoV-2 in a single tube. Further embodiments include consumables that contain primers, probes, and other reagents for the performance of the methods, and in which the methods may be performed. The detection methods may be designed to target various regions of each of the target genomes. For example, the methods may be designed to target one or more of the regions of the SARS-CoV-2 genome that encode the nucleoprotein (N) region, the non-structural Open Reading Frame (ORF1a/b) region, the S gene (coding for spike protein responsible for binding to cell receptor), ORF3ab, the E gene (coding for envelope protein), and the M gene (coding for membrane protein). In addition, there are 265 bases of non-coding region at the 5′ terminal end and 229 bases of non-coding region at the 3′ terminal end of the SARS-CoV-2 genome, and these may be targeted as well.

The present disclosure also provides methods for the rapid and simultaneous detection of the presence or absence of influenza A, influenza B, and SARS-CoV-2 in a biological or non-biological sample, for example, multiplex detection of influenza A, influenza B, and SARS-CoV-2, by qualitative or quantitative real-time reverse-transcription polymerase chain reaction (RT-PCR) in a single tube using a point of care (POC) device. As used herein, the terms “flu A” or “fluA” or variants thereof refer to influenza A; the terms “flu B” or “fluB” or variants thereof refer to influenza B. Embodiments include methods of detection of influenza A, influenza B, and SARS-CoV-2 comprising performing a reverse transcription step and at least one cycling step, which may include an amplifying step and a hybridizing step. Further embodiments include primers, probes, and kits that are designed for the detection of influenza A, influenza B, and/or SARS-CoV-2 in a single tube. Further embodiments include consumables that contain primers, probes, and other reagents for the performance of the methods, and in which the methods may be performed. The detection methods may be designed to target various regions of each of the target genomes. For example, the methods may be designed to target the regions of the SARS-CoV-2 genome noted above. An internal control primer and probe set may also be included to amplify the target region of an included internal control. Such an internal control may assist in monitoring the processing of the target virus through all steps of the assay process, and to help to detect the presence of possible inhibitors in the RT-PCR reactions.

With respect to influenza A and B, the methods may be designed to target any gene or non-coding regions within the eight segments that make up their genomes. For example, the methods may target a well-conserved region of the influenza A matrix gene, and/or a non-structural protein gene of influenza B.

Nucleic acid target amplification and detection may be accomplished via a reverse transcription polymerase chain reaction (RT-PCR). Given the infancy of the knowledge on the SARS-CoV-2 virus, a multi-target assay for the detection of conserved regions of the SARS-CoV-2 genome and select sequences within the region that are optimal for RT-PCR can be considered advantageous. Other regions may also be targeted to achieve desirable results, such as regions relevant for detection of specific SARS-CoV-2 and/or influenza viral variants.

The assay described herein may be performed on the Cobas® Liat® Analyzer (Roche Molecular Systems, Pleasanton, Calif.), which automates and integrates sample preparation and purification, nucleic acid amplification, and detection of the target sequence in biological samples. It is a point of care (POC) device that can provide test results for a variety of targets in 20 minutes or less, and thus is advantageous in setting where a rapid and accurate identification of a patient's infection is needed. Other than adding the sample to the assay tube, no reagent preparation or additional steps are required. The Cobas® Liat® Analyzer consists of an instrument and preloaded software for running tests and viewing the results. The system requires the use of a single-use disposable assay tube that holds the nucleic acid purification and RT-PCR reagents, and hosts the sample preparation and RT-PCR processes. The detection module monitors the reaction in real-time, while an on-board computer analyzes the collected data and displays an interpreted result. The latter is shown in the assay report on the integrated LCD touch screen of the Cobas® Liat® Analyzer and in an electronic file. The report can be printed directly through a USB or network-connected printer. The results can also be exported to an external server, middleware or data management system, or to a Laboratory Information System (LIS). See U.S. Pat. No. 6,780,617, the disclosure of which is incorporated in its entirety.

To perform the assay, a user collects e.g. saliva, nasopharyngeal, or nasal swab samples following the user institution's standard procedures. For samples suspended in viral transport media or physiological saline, a user transfers the sample into an assay tube using a transfer pipette. The operator then scans the assay tube barcode before inserting the assay tube into the Cobas® Liat® Analyzer. The assay tube is a plastic tube separated into segments delineated by frangible and burstable seals, and held taut on a rigid frame.

The sample preparation methodology is based on chaotropic agent-based lysis and magnetic glass particles-based (“MGP”) nucleic acid purification. First, a sample is diluted in a liquid transport medium and mixed with an internal control. Chaotropic and proteolytic lysis reagents then disrupt the three-dimensional structure of macromolecules (e.g., viral envelope proteins), and nucleic acids (e.g., viral genome) in the sample, and denature them. Second, nucleic acids are isolated from lysates through binding to the surface of MGPs in the presence of a chaotropic salt, which removes water from hydrated molecules in solution. Third, the MGPs are separated from the lysates using a magnetic field, and the lysate removed. Fourth, the beads with captured nucleic acids are washed to remove possible inhibitors in the sample. Finally, the captured nucleic acids are eluted under low-salt conditions into a small volume of elution buffer.

If the targeted nucleic acids comprise RNA, the eluted viral RNA is first reverse transcribed into complementary deoxyribonucleic acid (cDNA) using reverse transcriptase activity. If the targeted nucleic acids comprise DNA, no reverse transcriptase step is necessary. The DNA or cDNA then undergoes a polymerase chain reaction (“PCR”), where the reaction mixture is repeatedly heated to denature the nucleic acid and cooled to allow annealing of primers and extension of annealed primers by DNA polymerase to exponentially amplify one or more specific regions of DNA or cDNA. Dual labeled fluorogenic hydrolysis probes anneal to specific target sequences located between the binding regions of the forward and reverse primers. During the extension phase of the PCR cycle, the 5′ nuclease activity of the polymerase degrades the probes, causing the reporter dyes (e.g., 6-hexachlorocarboxyfluorescein (HEX)) to separate from the quenchers (e.g., Black Hole Quencher (BHQ)), thus generating fluorescent signals. Fluorescence intensities are monitored at each PCR cycle. When fluorescence intensities exceed pre-determined thresholds, cycle threshold (Ct) values are returned for the specific analyte corresponding to the fluorescence channel, in this case, SARS-CoV-2 and optionally influenza A and influenza B, plus an internal control (IC). Values for the relative maximal fluorescence signal measured (Amp) may also be calculated.

During the testing process, multiple sample processing actuators of the Cobas® Liat® Analyzer compress the assay tube to selectively open seals and release reagents from assay tube segments, move the sample from one segment to another, and control reaction conditions such as reaction volume, temperature, pressure, cycle number, and incubation time. The terms “actuator” and “compression member” are used interchangeably here. The actuators may be act as clamps, to apply pressure at the junctions between segments and hold selected segments closed. Precise control of all these parameters provides optimal conditions for assay reactions. An embedded microprocessor controls and coordinates these actions to perform all desired assay processes, including sample preparation, nucleic acid extraction, target concentration enrichment, inhibitor removal, nucleic acid elution, and real-time PCR.

All assay steps are performed within the closed and self-contained assay tube, minimizing the potential for cross-contamination between samples. The analyzer performs all test steps and displays interpreted results in approximately 20 minutes. A report of the interpreted results can be viewed in the View Results window, and printed directly through a USB connected printer.

In one embodiment, a method for detecting SARS-CoV-2 in a sample is provided, comprising performing an amplifying step including contacting the sample with at least one set of primers to produce one or more amplification products if SARS-CoV-2 is present in the sample; wherein the set of primers produces an amplification product if SARS-CoV-2 is present in the sample; performing a hybridizing step including contacting the amplification product(s) with one or more detectable probes, wherein the one or more detectable probes includes at least one probe specific for the amplification products of the at least one set of primers; and detecting the presence or absence of the amplified products, wherein the presence of the amplified product is indicative of the presence of SARS-CoV-2 in the sample and wherein the absence of the amplified product is indicative of the absence of SARS-CoV-2 in the sample.

In another embodiment, the at least one set of primers used in the method(s) comprises a first primer having an oligonucleotide sequence selected from the group consisting of SEQ ID NOs:1-3 or 7-9, and a second primer having an oligonucleotide sequence selected from the group consisting of SEQ ID NOs:4-6 or 13-15; and the one or more detectable probes comprises an oligonucleotide sequence selected from the group consisting of SEQ ID NOs:18-20.

In another embodiment, the at least one set of primers used in the method(s) comprises a first primer having an oligonucleotide sequence selected from the group consisting of SEQ ID NOs:1-3, and a second primer having an oligonucleotide sequence selected from the group consisting of SEQ ID NOs:4-6; and the detectable probe comprises an oligonucleotide sequence of SEQ ID NO:18.

In another embodiment, the at least one set of primers used in the method(s) comprises a first primer having an oligonucleotide sequence selected from the group consisting of SEQ ID NOs:10-12, and a second primer having an oligonucleotide sequence selected from the group consisting of SEQ ID NOs:16-17; and the detectable probe comprises an oligonucleotide sequence selected from the group consisting of SEQ ID NOs:21-22.

In another embodiment, the at least one set of primers used in the method(s) comprises a first primer having an oligonucleotide sequence of SEQ ID NO:3, and a second primer having an oligonucleotide sequence of SEQ ID NO:4; and the detectable probe comprises an oligonucleotide sequence of SEQ ID NO:18.

In another embodiment, the at least one set of primers used in the method(s) comprises a first primer having an oligonucleotide sequence of SEQ ID NO:12, and a second primer having an oligonucleotide sequence of SEQ ID NO:17; and the detectable probe comprises an oligonucleotide sequence of SEQ ID NO:22.

In another embodiment, the at least one set of primers used in the method(s) comprises a first primer set having an oligonucleotide sequence of SEQ ID NO:3 and a second primer having an oligonucleotide sequence of SEQ ID NO:4; a second primer set comprising a third primer having an oligonucleotide sequence of SEQ ID NO:12 and a fourth primer having an oligonucleotide sequence of SEQ ID NO:17; a first detectable probe comprising an oligonucleotide sequence of SEQ ID NO:18; and a second detectable probe comprising an oligonucleotide sequence of SEQ ID NO:22.

In another embodiment, a method for simultaneously detecting influenza A, influenza B, and SARS-CoV-2 in a sample is provided, comprising performing an amplifying step including contacting the sample with a first set of primers, a second set of primers, and a third set of primers, to produce one or more amplification products if influenza A, influenza B, and/or SARS-CoV-2 is present in the sample; wherein the first set of primers produces an amplification product if influenza A is present in the sample, the second set of primers produces an amplification product if influenza B is present in the sample, and the third set of primers produces an amplification product if SARS-CoV-2 is present in the sample; performing a hybridizing step including contacting the amplification product(s) with three or more detectable probes, wherein the three or more detectable probes includes at least one probe specific for the amplification products of each of the first, the second, and the third sets of primers; and detecting the presence or absence of the amplified products, wherein the presence of the amplified product is indicative of the presence of influenza A, influenza B, and/or SARS-CoV-2 in the sample and wherein the absence of the amplified product is indicative of the absence of influenza A, influenza B, and/or SARS-CoV-2 in the sample.

In another embodiment, the first set of primers used in the method(s) comprises a first primer having an oligonucleotide sequence selected from the group consisting of SEQ ID NOs:1-3, and a second primer having an oligonucleotide sequence selected from the group consisting of SEQ ID NOs:4-6; the second set of primers comprises a third primer having an oligonucleotide sequence of SEQ ID NO:23, and a fourth primer having an oligonucleotide sequence selected from the group consisting of SEQ ID NOs:24-25; and the third set of primers comprises a fifth primer having an oligonucleotide sequence of SEQ ID NO:28, and a sixth primer having an oligonucleotide sequence of SEQ ID NO:29; and wherein the first detectable probe comprises an oligonucleotide sequence of SEQ ID NO:18, the second detectable probe comprises an oligonucleotide sequence selected from the group consisting of SEQ ID NOs:26-27, and the third detectable probe comprises an oligonucleotide sequence of SEQ ID NO:30.

In other embodiments, a kit for detecting one or more of influenza A, influenza B, or SARS-CoV-2 is provided, the kit comprising at least a first set of primers, a second set of primers, and a third set of primers; and at least a first detectable probe, a second detectable probe, and a third detectable probe. In another embodiment, the kit may comprise a first set of primers comprising or consisting of a first primer having an oligonucleotide sequence selected from the group consisting of SEQ ID NOs:1-3, and a second primer comprising or consisting of an oligonucleotide sequence selected from the group consisting of SEQ ID NOs:4-6; a second set of primers comprising a third primer comprising or consisting of an oligonucleotide sequence of SEQ ID NO:23, and a fourth primer comprising or consisting of an oligonucleotide sequence selected from the group consisting of SEQ ID NOs:24-25; and a third set of primers comprising a fifth primer comprising or consisting of an oligonucleotide sequence of SEQ ID NO:28, and a sixth primer comprising or consisting of an oligonucleotide sequence of SEQ ID NO:29; and a first detectable probe comprising or consisting of an oligonucleotide sequence of SEQ ID NO:18, a second detectable probe comprising or consisting of an oligonucleotide sequence selected from the group consisting of SEQ ID NOs:26-27, and the third detectable probe comprising or consisting of an oligonucleotide sequence of SEQ ID NO:30. In other embodiments, the kit for detecting one or more of influenza A, influenza B, or SARS-CoV-2 may include, in addition to the primers and probes described above, a nucleic acid polymerase, dNTPs, and buffers that enable the activity of the nucleic acid polymerase. In certain embodiments, the nucleic acid polymerase is a DNA polymerase, and may be a thermostable DNA polymerase. In certain embodiments, the nucleic acid polymerase may be a thermostable DNA polymerase derived from a bacterium of the genus Thermus. The kit may also comprise a container and/or instructions, printed or stored on a computer-readable device, for using the kit components.

In other embodiments, a reaction vessel is provided, comprising a proximal end having an opening through which a sample is introducible; a distal end; and at least a first segment containing at least one nucleic acid extraction reagent, a second segment distal to the first segment and containing a wash reagent, and a third segment distal to the second segment and containing one or more amplification reagents, each of said segments being defined by the tubule and being fluidly isolated from other segments, at least in part, by a fluid-tight seal formed by a bonding of opposed wall portions of the tubule to one another such that the seal is broken by application of fluid pressure on a segment that is fluidly isolated in part by the seal; and the seal is capable of being clamped where the opposed wall portions of the tubule are bonded, without breaking the seal, to prevent the seal from being broken by application of fluid pressure on a segment that is fluidly isolated in part by the seal; so expandable as to receive a volume of fluid expelled from another segment; and so compressible as to contain substantially no fluid when so compressed; a cap for closing the opening, the cap containing a chamber in fluid communication with the tubule, and the cap permitting free escape of gasses but retaining all liquid volumes and infectious agents in the tube; a rigid frame to which the tubules proximal and distal ends are held; and an integral tubule tensioning mechanism or an attachment of the tubule to the frame that pulls the tubule sufficiently taut so as to facilitate compression and flattening of the tubule; the reaction vessel containing a first set of primers, a second set of primers, and a third set of primers; and at least a first detectable probe, a second detectable probe, and a third detectable probe; wherein the first set of primers comprises a first primer comprising or consisting of an oligonucleotide sequence selected from the group consisting of SEQ ID NOs:1-3, and a second primer comprising or consisting of an oligonucleotide sequence selected from the group consisting of SEQ ID NOs:4-6; the second set of primers comprises a third primer comprising or consisting of an oligonucleotide sequence of SEQ ID NO:23, and a fourth primer comprising or consisting of an oligonucleotide sequence selected from the group consisting of SEQ ID NOs:24-25; and the third set of primers comprises a fifth primer comprising or consisting of an oligonucleotide sequence of SEQ ID NO:28, and a sixth primer comprising or consisting of an oligonucleotide sequence of SEQ ID NO:29; and wherein the first detectable probe comprises or consists of an oligonucleotide sequence of SEQ ID NO:18, the second detectable probe comprises or consists of an oligonucleotide sequence selected from the group consisting of SEQ ID NOs:26-27, and the third detectable probe comprises or consists of an oligonucleotide sequence of SEQ ID NO:30.

In other embodiments, the methods, kits and reaction vessels disclosed herein may utilize a set of primers for simultaneous amplification of the influenza A, influenza B, and SARS-CoV-2 targets comprising or consisting of a first primer of SEQ ID NO:3, a second primer comprising or consisting of a second oligonucleotide sequence of SEQ ID NO:4, a third primer comprising or consisting of a third oligonucleotide sequence of SEQ ID NO:12, a fourth primer comprising or consisting of a fourth oligonucleotide sequence of SEQ ID NO:17; a fifth primer comprising or consisting of a fifth oligonucleotide sequence of SEQ ID NO: 23, a sixth primer comprising or consisting of a sixth oligonucleotide sequence of SEQ ID NO: 25, a seventh primer comprising or consisting of a seventh oligonucleotide sequence of SEQ ID NO:28, and an eighth primer comprising or consisting of a eighth oligonucleotide sequence of SEQ ID NO:29.

In other embodiments, the methods, kits and reaction vessels disclosed herein may utilize a set of primers for simultaneous amplification of the influenza A, influenza B, and SARS-CoV-2 targets comprising or consisting of a first primer of SEQ ID NO:3, a second primer comprising or consisting of a second oligonucleotide sequence of SEQ ID NO:4, a third primer comprising or consisting of a third oligonucleotide sequence of SEQ ID NO:12, a fourth primer comprising or consisting of a fourth oligonucleotide sequence of SEQ ID NO:17; a fifth primer comprising or consisting of a fifth oligonucleotide sequence of SEQ ID NO: 23, a sixth primer comprising or consisting of a sixth oligonucleotide sequence of SEQ ID NO: 24, a seventh primer comprising or consisting of a seventh oligonucleotide sequence of SEQ ID NO: 25; an eighth primer comprising or consisting of an eighth oligonucleotide sequence of SEQ ID NO: 28, and a ninth primer comprising or consisting of a ninth oligonucleotide sequence of SEQ ID NO:29.

In another embodiment, there is provided a method of detecting Severe Acute Respiratory Syndrome Coronavirus 2 (SARS-CoV-2) nucleic acid in a sample, comprising contacting the sample with at least a first set of primers and a second set of primers under conditions suitable for producing one or more amplification products if a target nucleic acid is present in the sample; contacting the sample with at least a first detectable probe and a second detectable probe under conditions suitable for producing a signal from at least one of the first and second detectable probes if one or more amplification products are present; and detecting the signal produced in step b), wherein the presence of the one or more amplification products is indicative of the presence of SARS-CoV-2 nucleic acids in the sample and wherein the absence of the one or more amplification products is indicative of the absence of SARS-CoV-2 nucleic acids in the sample; wherein the first set of primers comprises a first primer having an oligonucleotide sequence selected from the group consisting of SEQ ID NOs:1-3 and SEQ ID NOs: 7-9, and a second primer having an oligonucleotide sequence selected from the group consisting of SEQ ID NOs:4-6 and SEQ ID NOs: 13-15; and wherein the second set of primers comprises a third primer having an oligonucleotide sequence selected from the group consisting of SEQ ID NOs: 10-12, and a fourth primer having an oligonucleotide sequence selected from the group consisting of SEQ ID NOs:16-17; and wherein the first detectable probe comprises an oligonucleotide sequence selected from the group consisting of SEQ ID NOs:18-20, and the second detectable probe comprises an oligonucleotide sequence selected from the group consisting of SEQ ID NOs:21-22. The first primer can comprise an oligonucleotide sequence selected from the group consisting of SEQ ID NOs:1-3, and the second primer can comprise an oligonucleotide sequence selected from the group consisting of SEQ ID NOs: 4-6. In another embodiment, the method may be one wherein each of the at least a first detectable probe and a second detectable probe are labeled with a donor fluorescent moiety and a corresponding acceptor moiety; and step c) comprises detecting the presence or absence of fluorescence resonance energy transfer (FRET) between the donor fluorescent moiety and the acceptor moiety of the at least first and second detectable probes, wherein the presence or absence of fluorescence is indicative of the presence or absence of SARS-CoV-2 nucleic acids in the sample. In another embodiment, the method may be one wherein the donor fluorescent moiety and the corresponding acceptor moiety on each of said first and second detectable probes are separated by 8-20 nucleotides, inclusive. In another embodiment, the method may be one wherein each of said first and second detectable probes are labeled with a different donor fluorescent moiety selected from the group consisting of a fluorescein dye, a rhodamine dye, a cyanine dye, and a coumarin dye. In another embodiment, the method may be one wherein said donor fluorescent moieties on said first and second detectable probes are the same, and are selected from the group consisting of Cy2, Cy3, Cy5, Cy 5.5 and Cy7. In another embodiment, the method may be one wherein at least one of the primers and detectable probes includes a modified nucleotide. In another embodiment, the method may be one wherein said modified nucleotide is selected from the group consisting of a t-butyl benzyl, a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolopyrimidine analog, a pseudo-dU, a nitro pyrrole, a nitro indole, 2′-0-methyl ribo-U, 2′-0-methyl ribo-C, an N4-ethyl-dC, and an N6-methyl-dA. In another embodiment, the method may be one further comprising detecting a nucleic acid from one or more other viruses, and wherein the one or more other viruses is selected from the group consisting of influenza A, influenza B, influenza C, influenza D, respiratory syncytial virus (RSV), bat-coronavirus, severe acute respiratory syndrome (SARS) coronavirus (SARS-CoV), and Middle East respiratory syndrome (MERS) coronavirus (MERS-CoV). In another embodiment, the method may be one wherein the first primer comprises a sequence of SEQ ID NO:3 and the second primer comprises a sequence of SEQ ID NO:4. In another embodiment, the method may be one wherein the third primer comprises a sequence of SEQ ID NO:12 and the fourth primer comprises a sequence of SEQ ID NO:17. In another embodiment, the method may be one wherein the second detectable probe comprises a sequence of SEQ ID NO:22. In another embodiment, the method may further comprise a third set of primers, a fourth set of primers, a third detectable probe, and a fourth detectable probe; wherein the third set of primers comprises a fifth primer having an oligonucleotide sequence selected from the group consisting of SEQ ID NO:23 and SEQ ID NOs: 7-9, and a sixth primer having an oligonucleotide sequence selected from the group consisting of SEQ ID NOs:24-25; and wherein the fourth set of primers comprises a seventh primer having an oligonucleotide sequence consisting of SEQ ID NO:28 and an eighth primer having an oligonucleotide sequence consisting of SEQ ID NO:29; and wherein the third detectable probe comprises an oligonucleotide having a sequence selected from the group consisting of SEQ ID NOs: 26-27, and the fourth detectable probe comprises an oligonucleotide having a sequence consisting of SEQ ID NO:30; each of said third detectable probe and said fourth detectable probe being labeled with a different donor fluorescent moiety and a corresponding acceptor moiety; and step c) further comprises detecting the presence or absence of fluorescence resonance energy transfer (FRET) between the donor fluorescent moiety and the acceptor moiety of the third and fourth detectable probes, wherein the presence or absence of fluorescence from the third detectable probe is indicative of the presence or absence of influenza A nucleic acids in the sample, and wherein the presence or absence of fluorescence from the fourth detectable probe is indicative of the presence or absence of influenza B nucleic acids in the sample. In another embodiment, each of said third and fourth detectable probes may be labeled with a different donor fluorescent moiety selected from the group consisting of a fluorescein dye, a rhodamine dye, a cyanine dye, and a coumarin dye. In another embodiment, the method may be one wherein a donor fluorescent moiety is located on a terminal nucleotide of at least one of said first, second, third, and fourth detectable probes, and the corresponding acceptor moiety is located on the other terminal nucleotide of at least one of said first, second, third, and fourth detectable probes.

In another embodiment, there is provided a kit for detecting SARS-CoV-2, comprising a first set of primers and a second set of primers; a first detectable probe and a second detectable probe; the first set of primers comprising a first primer having an oligonucleotide sequence selected from the group consisting of SEQ ID NOs:1-3 and SEQ ID NOs: 7-9, and a second primer having an oligonucleotide sequence selected from the group consisting of SEQ ID NOs:4-6 and SEQ ID NOs: 13-15; the second set of primers comprising a third primer having an oligonucleotide sequence of SEQ ID NOs: 10-12, and a fourth primer having an oligonucleotide sequence selected from the group consisting of SEQ ID NOs:16-17; and the first detectable probe comprising an oligonucleotide sequence of SEQ ID NOs:18-20; and the second detectable probe comprising an oligonucleotide sequence selected from the group consisting of SEQ ID NOs:21-22. In another embodiment, the kit may further comprise a third set of primers, a fourth set of primers, a third detectable probe, and a fourth detectable probe; wherein the third set of primers comprises a fifth primer having an oligonucleotide sequence selected from the group consisting of SEQ ID NO:23 and SEQ ID NOs: 7-9, and a sixth primer having an oligonucleotide sequence selected from the group consisting of SEQ ID NOs:24-25; and wherein the fourth set of primers comprises a seventh primer having an oligonucleotide sequence consisting of SEQ ID NO:28 and an eighth primer having an oligonucleotide sequence consisting of SEQ ID NO:29; and wherein the third detectable probe comprises an oligonucleotide having a sequence selected from the group consisting of SEQ ID NOs: 26-27, and the fourth detectable probe comprises an oligonucleotide having a sequence consisting of SEQ ID NO:30.

In another embodiment there is provided a reaction vessel, comprising a proximal end having an opening through which a sample is introducible; a distal end; and at least a first segment containing at least one nucleic acid extraction reagent, a second segment distal to the first segment and containing a wash reagent, and a third segment distal to the second segment and containing one or more amplification reagents, each of said segments being defined by the tubule; fluidly isolated, at least in part, by a fluid-tight seal formed by a bonding of opposed wall portions of the tubule to one another such that the seal is broken by application of fluid pressure on a segment that is fluidly isolated in part by the seal; and the seal is capable of being clamped where the opposed wall portions of the tubule are bonded, without breaking the seal, to prevent the seal from being broken by application of fluid pressure on a segment that is fluidly isolated in part by the seal; so expandable as to receive a volume of fluid expelled from another segment; and so compressible as to contain substantially no fluid when so compressed; a cap for closing the opening, the cap containing a chamber in fluid communication with the tubule, and the cap permitting free escape of gasses but retaining all liquid volumes and infectious agents in the tube; a rigid frame to which the tubules proximal and distal ends are held; and an integral tubule tensioning mechanism or an attachment of the tubule to the frame that pulls the tubule sufficiently taut so as to facilitate compression and flattening of the tubule; said reaction vessel containing a first set of primers and a second set of primers; a first detectable probe and a second detectable probe; the first set of primers comprising a first primer having an oligonucleotide sequence selected from the group consisting of SEQ ID NOs:1-3 and SEQ ID NOs: 7-9, and a second primer having an oligonucleotide sequence selected from the group consisting of SEQ ID NOs:4-6 and SEQ ID NOs: 13-15; the second set of primers comprising a third primer having an oligonucleotide sequence of SEQ ID NOs: 10-12, and a fourth primer having an oligonucleotide sequence selected from the group consisting of SEQ ID NOs:16-17; and the first detectable probe comprising an oligonucleotide sequence of SEQ ID NOs:18-20; and the second detectable probe comprising an oligonucleotide sequence selected from the group consisting of SEQ ID NOs:21-22.

Generally, the oligonucleotides may be primer nucleic acids, probe nucleic acids, or the like. The oligonucleotides may include one or more nucleic acids having at least 70% sequence identity (e.g., at least 75%, 80%, 85%, 90% or 95%, etc.) to one of SEQ ID NOs:1-30. The oligonucleotides may have 100 or fewer nucleotides. In certain of these embodiments, the oligonucleotides have 40 or fewer nucleotides (e.g., 35 or fewer nucleotides, 30 or fewer nucleotides, 25 or fewer nucleotides, 20 or fewer nucleotides, 15 or fewer nucleotides, etc.) In some embodiments, the oligonucleotides comprise at least one modified nucleotide, e.g., to alter nucleic acid hybridization stability and/or specificity relative to unmodified nucleotides.

In certain embodiments, amplification can be achieved using a nucleic acid polymerase enzyme having 5′ to 3′ nuclease activity. The amplification may be achieved using a polymerase chain reaction (PCR). In some embodiments, where the target comprises an RNA template rather than a DNA template, amplification may be achieved using a reverse-transcription PCR (RT-PCR). In some embodiments, where quantification of the amount of the target in a sample is desirable, a quantitative RT-PCR (qRT-PCR) may be employed.

In certain embodiments, the oligonucleotides acting as probes comprise at least one donor fluorescent moiety and at least one acceptor moiety. The acceptor moiety may itself be fluorescent, or alternately, may be a dark quencher. The donor fluorescent moiety and the acceptor moiety may be within 8 to 20 nucleotides of each other along the length of the probe. In another aspect, the probe includes a nucleic acid sequence that permits secondary structure formation. Such secondary structure formation may result in spatial proximity between the first and second fluorescent moiety. Fluorescence from the reporter dye or the acceptor dye is measured at a defined wavelength, thus permitting detection and discrimination of the amplified targets. Initially, the fluorescent signal of the intact probe is suppressed by the quencher dye. During the PCR amplification step, hybridization of the probe to the specific single-stranded DNA template results in cleavage by the 5′ to 3′ nuclease activity of the nucleic acid polymerase, resulting in separation of the reporter and quencher dyes and the generation of a fluorescent signal. With each PCR cycle, increasing amounts of cleaved probes are generated and the cumulative signal of the reporter dye is concomitantly increased. Optionally, one or more additional probes (e.g., such as an internal reference control) may also be labeled with a reporter fluorescent dye, unique and distinct from the fluorescent dye label associated with the target probes. In such a case, because the specific reporter dyes are measured at defined wavelengths, simultaneous detection and discrimination of the amplified targets and the one or more additional probes is possible.

The methods of detecting the presence or absence of SARS-CoV-2, or SARS-CoV-2 nucleic acids, or influenza A, influenza B, and SARS-CoV-2, or influenza A, influenza B, and SARS-CoV-2 nucleic acids, disclosed herein may be applied to a biological sample from a human individual. Such biological samples may be obtained from patients using sampling methods known to those in the art, e.g., a nasal swab sample, a throat swab sample, a nasal mid-turbinate swab sample, a nasopharyngeal wash/aspirate sample, a nasal wash/aspirate sample, a nasopharyngeal swab sample, or an oropharyngeal swab sample. Additionally, the same test may be used by those experienced in the art to assess other sample types (e.g., sputum, saliva, blood, urine, feces, oral fluid, lower respiratory tract aspirate, bronchoalveolar lavage fluid, pleural fluid, lung biopsy, or derivatives thereof, etc.) to detect SARS-CoV-2, or simultaneously detect influenza A, influenza B, and SARS-CoV-2, or influenza A, influenza B, and SARS-CoV-2 nucleic acids. The methods disclosed herein may also be applied to non-biological samples (e.g., water samples, air samples, food samples, agricultural samples, environmental samples, soil samples, liquid testing samples, surface testing samples, etc.), or to biological samples obtained from a non-human source, e.g., livestock, wild animals, domesticated animals, etc.

In one aspect, a kit can include probes labeled with donor and corresponding acceptor moieties, e.g., another fluorescent moiety or a dark quencher, or can include fluorophoric moieties for labeling oligonucleotides. The kit can also include nucleoside triphosphates, nucleic acid polymerase, and buffers necessary for the function of the nucleic acid polymerase. The kit can also include a package insert and instructions for using the primers, probes, and fluorophoric moieties to detect the presence or absence of influenza A, influenza B, and SARS-CoV-2 nucleic acid in a sample.

The reaction vessels and kits described herein are suitable for use with a device for processing a sample that includes a processing unit e.g., an analyzer, having an opening to receive a consumable (also known as a sample vessel) and at least one processing station positioned along the opening. See U.S. Pat. Nos. 7,718,421; 6,780,617; and 6,748,617, each of which are hereby incorporated by reference in its entirety. The terms “sample vessel” and “consumable” are used herein interchangeably. The processing unit includes at least one compression member (or actuator) adapted to compress the sample vessel within the opening and thereby displace a content of the sample vessel within the sample vessel. Other compression members may act as clamps, to hold a certain volume of the sample vessel contents at a defined location. The processing unit further includes at least one energy transfer element, which may transfer thermal energy to or from the content within the sample vessel.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 shows the genome organization of SARS-CoV-2 (labeled here as Wuhan-Hu-1) and SARS-CoV and the locations of the target regions of the SARS-CoV-2 primer and probes described herein. E: envelope protein gene; M: membrane protein gene; N: nucleocapsid protein gene; ORF1a/b: ORF for non-structural genes; S: spike protein gene. Numbers below the amplicon are genome position according to Wuhan-Hu-1, GenBank MN908947.

FIG. 2 is a partial representation of the genome organizations of the influenza A and influenza B viruses, and the locations of the oligonucleotide sets used herein. FIG. 2A: partial view of genome organization of influenza A. Reference: KC781450 influenza A virus (A/Michigan/01/2010(H1N1)) segment 7 matrix protein 2 (M2) and matrix protein 1 (M1) genes. FIG. 2B: partial view of genome organization of influenza B. Reference: KM654608 influenza B virus (B/Connecticut/Flu103/2013) segment 8 nuclear export protein (NEP) and nonstructural protein 1 (NS1) genes.

FIG. 3 shows performance of various oligonucleotide sets disclosed herein (Tables 2-4) for use in an assay to simultaneously detect influenza A, influenza B, and SARS-CoV-2 (orflab target). Ct (or cycle threshold) is the number of PCR cycles required for the fluorescent signal to cross a pre-determined signal threshold. Amp is the calculated average of the final amplitude of the PCR growth curve. FIG. 3A: detection of influenza A RNA target in presence of various SARS-CoV-2 orflab oligonucleotide sets—Ct measurement. FIG. 3B: detection of influenza A RNA target in presence of various SARS-CoV-2 orflab oligonucleotide sets—Amp measurement.

FIG. 3C: detection of influenza B target in presence of various SARS-CoV-2 orflab oligonucleotide sets—Ct measurement. FIG. 3D: detection of influenza B RNA target in presence of various SARS-CoV-2 orflab oligonucleotide sets—Amp measurement. FIG. 3E: detection of nCoV1 transcript in presence of various SARS-CoV-2 orflab oligonucleotide sets—Ct measurement. FIG. 3F: detection of nCoV1 transcript in presence of various SARS-CoV-2 orflab oligonucleotide sets—Amp measurement. The diamonds show the distribution of Ct and Amp values. The middle horizontal line shows the average value, the upper line shows top 75%, and the lower line shows the bottom 25%.

FIG. 4 shows performance of selected SARS-CoV-2 orflab oligonucleotide sets in detecting a standard SARS-CoV-2 sequence (AccuPlex™ SARS-CoV-2 Verification Panel Member 1, SeraCare, Milford, Mass., USA) (“AccuPlex™ Panel Member 1”) in a synthetic sample. The synthetic sample contained influenza A (4.92×10⁻³ TC1D₅₀/mL), influenza B (1.67×10⁻³ TC1D₅₀/mL) and 400 copies/mL of AccuPlex™ Panel Member 1 in simulated Universal Transport Media (“sUTM”). Concentrations were determined by the vendor. sUTM was made by mixing Universal Transport Medium (Copan Diagnostics, Inc., Murrieta, Calif.) with human epithelial cells and porcine mucin. FIG. 4A: detection of influenza A RNA target in presence of SARS-CoV-2 orflab oligonucleotide sets—Ct measurement. FIG. 4B: detection of influenza A RNA target in presence of SARS-CoV-2 orflab oligonucleotide sets—Amp measurement. FIG. 4C: detection of influenza B target in presence of SARS-CoV-2 orflab oligonucleotide sets—Ct measurement. FIG. 4D: detection of influenza B RNA target in presence of SARS-CoV-2 orflab oligonucleotide sets—Amp measurement. FIG. 4E: detection of AccuPlex™ Panel Member 1 in presence of SARS-CoV-2 orflab oligonucleotide sets—Ct measurement. FIG. 4F: detection of AccuPlex™ Panel Member 1 in presence of SARS-CoV-2 orflab oligonucleotide sets—Amp measurement. The diamonds show the distribution of Ct and Amp. The middle horizontal line shows the average value, the upper line shows top 75%, and the lower line shows the bottom 25%.

FIG. 5 shows performance of various candidate SARS-CoV-2 N gene oligonucleotide sets in detecting a standard SARS-CoV-2 sequence (AccuPlex™ Panel Member 1) in a synthetic sample. The synthetic sample contained influenza A (4.92×10⁻³ TC1D₅₀/mL), influenza B (1.67×10⁻³ TC1D₅₀/mL) and 400 copies/mL of AccuPlex™ Panel Member 1 in simulated Universal Transport Media. FIG. 5A: detection of influenza A RNA target in presence of SARS-CoV-2 N gene oligonucleotide sets—Ct measurement. FIG. 5B: detection of influenza A RNA target in presence of SARS-CoV-2 N gene oligonucleotide sets—Amp measurement. FIG. 5C: detection of influenza B RNA target in presence of SARS-CoV-2 N gene oligonucleotide sets—Ct measurement. FIG. 5D: detection of influenza B RNA target in presence of SARS-CoV-2 N gene oligonucleotide sets—Amp measurement. FIG. 5E: detection of AccuPlex™ Panel Member 1 in presence of SARS-CoV-2 N gene oligonucleotide sets—Ct measurement. FIG. 5F: detection of AccuPlex™ Panel Member 1 in presence of SARS-CoV-2 N gene oligonucleotide sets—Amp measurement. The diamonds show the distribution of Ct and Amp. The middle horizontal line shows the average value, the upper line shows top 75%, and the lower line shows the bottom 25%.

FIG. 6 shows the performance of the SARS-CoV-2 single target and dual target assays against that of the A/B-RSV test test in sUTM, in detection of influenza A and influenza B targets. STA=single target assay; DTA=dual target assay. FIG. 6A: detection of influenza A RNA target—Ct measurement. FIG. 6B: detection of influenza A RNA target—Amp measurement. FIG. 6C: detection of influenza B RNA target—Ct measurement. FIG. 6D: detection of influenza B RNA target—Amp measurement. The diamonds show the distribution of Ct and Amp. The middle horizontal line shows the average value, the upper line shows top 75%, and the lower line shows the bottom 25%.

FIG. 7 shows the performance of the SARS-CoV-2 single target and dual target assays against that of the A/B-RSV test in UTM (Copan Diagnostics, Inc., Murrieta, Calif.), in detection of influenza A and influenza B targets. STA=single target assay; DTA=dual target assay. FIG. 7A: detection of influenza A RNA target—Ct measurement. FIG. 7B: detection of influenza A RNA target—Amp measurement. FIG. 7C: detection of influenza B RNA target—Ct measurement. FIG. 7D: detection of influenza B RNA target—Amp measurement. The diamonds show the distribution of Ct and Amp. The middle horizontal line shows the average value, the upper line shows top 75%, and the lower line shows the bottom 25%.

FIG. 8 shows the performance of the SARS-Cov-2 single target and dual target assays in detection of Accuplex™ Panel Member 1 in sUTM. FIG. 8A: detection of Accuplex™ Panel Member 1 (50 copies/ml and 100 copies/ml) in SARS-CoV-2 STA and DTA assays—Ct measurement. FIG. 8B: detection of Accuplex™ Panel Member 1 (50 copies/ml and 100 copies/ml) in SARS-CoV-2 STA and DTA assays—Amp measurement. The diamonds show the distribution of Ct and Amp. The middle horizontal line shows the average value.

FIG. 9 shows the performance of the SARS-CoV-2 single target and dual target assays in detection of Accuplex™ Reference Material (SeraCare, Milford, Mass., USA). FIG. 9A: detection of Accuplex™ Reference Material in UTM (25 copies/ml and 50 copies/ml) in SARS-CoV-2 STA and DTA assays—Ct measurement. FIG. 9B: detection of Accuplex™ Reference Material in UTM (25 copies/ml and 50 copies/ml) in SARS-CoV-2 STA and DTA assays—Amp measurement. The diamonds show the distribution of Ct and Amp. The middle horizontal line shows the average value.

FIG. 10 is a table showing the results of studies of competitive inhibition between influenza A, influenza B, and SARS-CoV-2, using the methods disclosed herein.

### DETAILED DESCRIPTION OF THE INVENTION

Diagnosis of SARS-CoV-2 infection by nucleic acid amplification provides a method for rapidly, accurately, reliably, specifically, and sensitively detecting the viral infection. A real-time reverse-transcriptase PCR assay for detecting SARS-CoV-2 in a non-biological or biological sample in a single tube using a point of care (POC) device is described herein. Primers and probes for detecting SARS-CoV-2 are provided, as are articles of manufacture or kits containing such primers and probes. The increased specificity and sensitivity of real-time PCR for detection of SARS-CoV-2 compared to other methods, as well as the improved features of real-time PCR including sample containment and real-time detection of the amplified product, make feasible the implementation of this technology for routine diagnosis of SARS-CoV-2 infections in a POC setting. This SARS-CoV-2 detection assay may also be multiplexed with other assays for the detection of other nucleic acids, e.g., influenza virus, SARS-CoV, MERS-CoV, in parallel.

In addition, simultaneous diagnosis of influenza A, influenza B, and SARS-CoV-2 infection by nucleic acid amplification provides a method for rapidly, accurately, reliably, specifically, and sensitively detecting and differentiating these respiratory viral infections. A real-time reverse-transcriptase PCR assay in a single tube using a point of care (POC) device for detecting and differentiating influenza A, influenza B, and SARS-CoV-2 in a non-biological or biological sample is described herein. Primers and probes for detecting influenza A, influenza B, and SARS-CoV-2 are provided, as are articles of manufacture or kits containing such primers and probes. The increased specificity and sensitivity of real-time PCR for detection of influenza A, influenza B, and SARS-CoV-2 compared to other methods, as well as the improved features of real-time PCR including sample containment and real-time detection of the amplified products, make feasible the implementation of this technology for routine diagnosis of influenza A, influenza B, and SARS-CoV-2 infections in a POC setting. Additionally, this technology may be employed for in vitro diagnostics as well as for prognosis. The assay is useful for detection of infection of both symptomatic and asymptomatic individuals. This SARS-CoV-2 detection multiplex assay may also be further multiplexed with other assays for the detection of other viral targets, including but not limited to influenza C virus, influenza D virus, SARS-1, MERS, or other coronaviruses, in parallel. As well, the assay may be performed with only the oligonucleotides for one or some of the targets. In such a case, the primers and/or probes for the other target(s) may be removed. So, for example, an assay for SARS-CoV-2 may be performed after removing or neutralizing the primers and/or probe(s) for influenza A and influenza B; or an assay to detect influenza A and SARS-CoV-2 may be performed after removing or neutralizing the primers and/or probe(s) for influenza B; or an assay to detect influenza B and SARS-Cov-2 may be performed after removing or neutralizing the primers and/or probe(s) for influenza A.

The present disclosure includes oligonucleotide primers and fluorescent labeled hydrolysis probes that hybridize to the SARS-CoV-2 genome (e.g., at the ORF1ab gene and/or at the N gene), in order to specifically identify SARS-CoV-2 using, e.g., RT-PCR or qRT-PCR amplification and detection technology. The oligonucleotides specifically hybridize to the ORF1ab gene, and/or to the N gene. Having oligonucleotides that hybridize to multiple locations in the genome is advantageous for improved sensitivity compared to targeting a single copy genetic locus.

Where the target viruses are RNA viruses, the disclosed methods also include performing a reverse transcription step, in addition to at least one cycling step that includes amplifying one or more portions of the nucleic acid molecule gene target from a sample using one or more pairs of primers.

The term “sample” as used herein includes any specimen or culture (e.g., microbiological cultures) that includes nucleic acids. The term “sample” is also meant to include both biological and non-biological samples. A “biological sample”, as used herein, generally refers to a sample derived from a living organism, including a viral organism. Examples of biological samples include whole blood, serum, plasma, umbilical cord blood, chorionic amniotic fluid, cerebrospinal fluid, spinal fluid, lavage fluid (e.g., bronchioalveolar, gastric, peritoneal, ductal, ear, arthroscopic), biopsy sample, urine, feces, sputum, saliva, nasal mucous, prostate fluid, semen, lymphatic fluid, bile, tears, sweat, breast milk, breast fluid, embryonic cells and fetal cells. In the context of the present disclosure, a biological sample may particularly refer to a nasal swab sample, a throat swab sample, a nasal mid-turbinate swab sample, a nasopharyngeal wash/aspirate sample, a nasal wash/aspirate sample, a nasopharyngeal swab sample, an oropharyngeal swab sample, sputum, blood, urine, feces, oral fluid, lower respiratory tract aspirate, bronchoalveolar lavage fluid, pleural fluid, lung biopsy, or derivatives thereof, etc.) Non-biological samples include environmental material such as surface matter, soil, water and industrial samples, as well as samples obtained from food and dairy processing instruments, apparatus, equipment, utensils, disposable and non-disposable items, liquid testing samples, surface testing samples, etc.

As used herein, the term “tube” refers to a cylindrical vessel made from a flexible plastic having an open end and a closed end. The open end may be held closed by a cap, and the tube held in a predetermined orientation by a rigid frame. The term “tube” may be used interchangeably with “reaction vessel” or like terms herein. The flexible plastic tube may be divided into segments separated by seals. The seals are burstable, that is, they are constructed so that upon application of a predetermined pressure, the seals open, allowing the liquid contents of one or more adjacent segments to mix. As used herein, the terms “segments” and “compartments” are used interchangeably. Also as used herein, the terms “burstable”, “frangible”, and “breakable” are interchangeable when used in reference to the character of the seals.

As used herein, the term “amplifying” refers to the process of synthesizing nucleic acid molecules that are copies of or complementary to one or both strands of a template nucleic acid molecule (e.g., nucleic acid molecules from the influenza A, influenza B, or SARS-CoV-2 genomes). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq, Thermo Fisher, Waltham, Mass., USA) and an appropriate buffer and/or co-factors for optimal activity of the polymerase enzyme (e.g., MgCl₂ and/or KCl). An “amplification product” as used herein refers to the nucleic acid products produced by the amplifying procedure.

As used herein, the term “hybridizing” refers to the annealing of one or more primers and/or probes to a nucleic acid template or to an amplification product. “Hybridization conditions” typically include a temperature that is below the melting temperature of the primers and/or probes, but that avoids non-specific, e.g., sequence-independent, hybridization.

As used herein, the term “detecting” means discovery or determination of the presence, absence, level or quantity, as well as a probability or likelihood of the presence or absence of a nucleic acid sequence. A “detectable moiety”, “reporter”, “fluorophore”, or “label”, is a molecule that confers a detectable signal and acts as a reporter, i.e., it signals the presence, absence, level or quantity of a specific oligonucleotide target molecule. The detectable signal can be colorimetric, fluorescent or luminescent, for example. Especially useful labels for oligonucleotide detection are fluorescent dyes, e.g., a fluorescein dye, a rhodamine dye, a cyanine dye, a coumarin dye, or a dye of the BODIPY®-family dyes (Thermo Fisher Scientific, Waltham, Mass., USA). Dyes of the fluorescein family include, e.g., 5,6-carboxyfluorescein (FAM), 2′,4,4′,5′,7,7′-hexachlorofluorescein (HEX), tetrachlorofluorescein (TET), and 6-carboxy-4′,5′-dichloro-2′,7′-dimethoxyfluorescein (JOE). Dyes of the rhodamine family include, e.g., Texas Red® (Thermo Fisher Scientific, Waltham, Mass., USA), carboxy-rhodamine (ROX), Rhodamine Green™, Rhodamine Red™, Rhodamine 6G, carboxytetramethyl-rhodamine (TAMRA), as well as the rhodamine derivative JA270 (see, U.S. Pat. No. 6,184,379, issued Feb. 6, 2001, to Josel et al.). Dyes of the cyanine family include, e.g., Cy2, Cy3, Cy5, Cy 5.5 and Cy7, and variants thereof. The fluorescein dyes, rhodamine dyes, and cyanine dyes listed above are generally commercially available from a variety of sources, e.g., Thermo Fisher Scientific (Waltham, Mass.). Other useful fluorescent dyes are e.g., Biosearch Blue™, Quasar™ 570, Quasar™ 670, Quasar™ 705, Pulsar™ 650, LC-Red 640, LC-Red 705, and other dyes. Other useful fluorescent dyes include those of the CAL Fluor® family of dyes, including CAL Fluor® Gold 540, CAL Fluor® Orange 560, CAL Fluor® Red 590, CAL Fluor Red® 610, and CAL Fluor® Red 635. The Biosearch Blue™, Quasar™, Pulsar™, and CAL Fluor family of dyes are available from LGC Biosearch Technologies, Novato, Calif.

The term “differentiating” refers to the ability to assess which of a plurality of specific targets is present in a sample.

A “quencher moiety” or “quencher molecule” is a molecule that is able to quench the detectable signal from the detectable moiety. Examples of quencher moieties used with fluorescent detectable moieties include, e.g., the so-called dark quenchers, such as Black Hole Quenchers® (BHQ®-1 or BHQ®-2) (LGC BioSearch Technologies, Novato, Calif.) or Iowa Black® (Integrated DNA Technologies, Coralville, Iowa); and fluorescent moities that use fluorescence resonance energy transfer (“FRET”), such as the cyanine dyes noted above. “Dark quenchers” are molecules that are capable of fluorescent excitation but instead of emitting light in response to excitation, they convert the excitation energy to heat.

FRET technology (see, for example, U.S. Pat. Nos. 4,996,143, 5,565,322, 5,849,489, and 6,162,603) is based on a concept that when a donor detectable moiety and a corresponding acceptor quencher moiety are positioned within a certain distance of each other, energy transfer takes place between the two moieties that can be visualized or otherwise detected and/or quantitated. The donor typically transfers the energy to the acceptor when the donor is excited by light radiation of a suitable wavelength. The acceptor typically re-emits the transferred energy in the form of either heat (dark quencher) or light radiation of a different wavelength. In certain systems, non-fluorescent energy can be transferred between donor and acceptor moieties, by way of biomolecules that include substantially non-fluorescent donor moieties (see, for example, U.S. Pat. No. 7,741,467).

In one example, an oligonucleotide probe can contain a donor fluorescent moiety (e.g., HEX dye) and a corresponding dark quencher (e.g., a BlackHole Quencher® (BHQ)). When the probe is intact, energy transfer typically occurs between the donor and acceptor moieties such that fluorescent emission from the donor fluorescent moiety is quenched by the acceptor moiety. During an extension step of a PCR, a probe bound to an amplification product is cleaved by the 5′ to 3′ nuclease activity of, e.g., a DNA polymerase, such that the fluorescent emission of the donor fluorescent moiety is no longer quenched and light is emitted at a wavelength characteristic of the detectable label. Exemplary probes for this purpose are described in, e.g., U.S. Pat. Nos. 5,210,015, 5,994,056, and 6,171,785.

Fluorescent detection can be carried out using, for example, a photon counting epifluorescent microscope system (containing the appropriate dichroic mirror and filters for monitoring fluorescent emission at the particular range), a photon counting photomultiplier system, or a fluorimeter. Excitation to initiate energy transfer, or to allow direct detection of a fluorophore, can be performed with an argon ion laser, a high intensity mercury (Hg) arc lamp, a xenon lamp, a fiber optic light source, or other high intensity light source appropriately filtered for excitation in the desired range.

The donor and acceptor fluorescent moieties may be attached to the appropriate probe oligonucleotide via a linker arm. The length of each linker arm is important, as the linker arms will affect the distance between the donor and acceptor fluorescent moieties. In general, a linker arm is from about 10 Å to about 25 Å in length, measured as the distance from the nucleotide base to the fluorescent moiety. The linker arm may be of the kind described in WO 84/03285. WO 84/03285 also discloses methods for attaching linker arms to a particular nucleotide base, and for attaching fluorescent moieties to a linker arm. Frequently used linkers to couple a donor or acceptor fluorescent moiety to an oligonucleotide include thiourea linkers (FITC-derived, for example, fluorescein-CPG's from Glen Research (Sterling, Va.) or ChemGene (Ashland, Mass.)), amide-linkers (fluorescein-NHS-ester-derived, such as CX-fluorescein-CPG from BioGenex (San Ramon, Calif.)), or 3′-amino-CPGs that require coupling of a fluorescein-NHS-ester after oligonucleotide synthesis.

The term “primer” as used herein refers to oligomeric nucleic acid compounds, primarily to oligonucleotides but also to modified oligonucleotides, that are able to “prime” DNA synthesis by a template-dependent nucleic acid polymerase, e.g., a DNA nucleic acid polymerase. The 3′-end of the oligonucleotide provides a free 3′—OH group where further nucleotides may be attached by a template-dependent nucleic acid polymerase. The primer hybridizes to a specific sequence of a single-stranded DNA target, and is extended by the nucleic acid polymerase through addition of nucleotides complementary to the target DNA molecule. A primer may also serve to prime a reverse transcription step, e.g., a template-dependent extension from a target RNA molecule to generate a cDNA molecule. A primer may be purified from a restriction digest by conventional methods, or it can be produced synthetically.

The term “probe” as used herein refers to an oligonucleotide that comprises at least one detectable moiety and that is used in a 5′-nuclease reaction to effect target nucleic acid detection. In some embodiments, for example, a probe includes only a single detectable moiety (e.g., a fluorescent dye, etc.) In certain embodiments, probes include regions of self-complementarity such that the probes are capable of forming hairpin structures under selected conditions. In some embodiments, a probe comprises at least two detectable moieties and emits radiation of increased intensity after one of the two labels is cleaved or otherwise separated from the oligonucleotide. In certain embodiments, a probe is labeled with two different fluorescent dyes, e.g., a 5′ terminal reporter dye and a 3′ terminal dye. In other embodiments, a probe is labeled with a fluorescent dye and a quencher, e.g., with a 5′ terminal reporter dye and a 3′ terminal quencher. In some embodiments, probes may be labeled with a quencher moiety at a position other than, or in addition to, a terminal position, e.g., the quencher may be located at an internal position. When the probe is intact, energy transfer typically occurs between the two fluorophores via fluorescence resonance energy transfer such that fluorescent emission from the reporter dye is reduced, or quenched, at least in part. During an extension step of a PCR, for example, a 5′-nuclease probe hybridized to a template nucleic acid is cleaved by the 5′ to 3′ nuclease activity of, e.g., a DNA polymerase or another polymerase having this activity, such that the fluorescent emission of the reporter dye is no longer quenched, and light is emitted from the reporter dye at its characteristic wavelength. Exemplary probes are described in, e.g., U.S. Pat. Nos. 5,210,015; 5,994,056; and 6,171,785.

Depending on the embodiment, the probe(s) used may comprise at least one label and optionally at least one quencher moiety. In some embodiments, a probe may be labeled with two or more different reporter dyes and a quencher dye or moiety. As with the primers, the probes usually have similar melting temperatures, and the length of each probe must be sufficient for sequence-specific hybridization to occur but not so long that fidelity is reduced during synthesis. Oligonucleotide primers and probes are generally 15 to 40 (e.g., 16, 18, 20, 21, 22, 23, 24, or 25) nucleotides in length.

As used herein, the term “SARS-CoV-2 primer(s)” refers to oligonucleotide primers that specifically anneal to nucleic acid sequences found in the SARS-CoV-2 genome, and initiate DNA synthesis therefrom under appropriate conditions producing the respective amplification products. Examples of nucleic acid sequences found in the SARS-CoV-2 genome include nucleic acids within the ORF1ab gene, the S gene, the ORF3ab gene, the E gene, the M gene, the N gene, and other predicted ORF regions as well as non-coding regions. Each of the discussed SARS-CoV-2 primers anneals to a target region such that at least a portion of each amplification product contains a nucleic acid sequence corresponding to the target. The one or more amplification products are produced if one or more nucleic acids that include the corresponding target sequence are present in the sample; in other words, the presence of the one or more amplification products is indicative of the presence of SARS-CoV-2 in the sample. The amplification product should contain the nucleic acid sequences that are complementary to one or more detectable probes for SARS-CoV-2. “SARS-CoV-2 probe(s)” as used herein refer to oligonucleotide probes that specifically anneal to nucleic acid sequences found in the SARS-CoV-2 genome. Each cycling step includes an amplification step, a hybridization step, and a detection step, in which the sample is contacted with the one or more detectable SARS-CoV-2 probes for detection of the presence or absence of SARS-CoV-2 in the sample.

Similarly, the terms “influenza A primer(s)” and “influenza B primer(s)” as used herein refer to oligonucleotide primers that specifically anneal to nucleic acid sequences found in the influenza A genome and the influenza B genome, respectively, and initiate DNA synthesis therefrom under appropriate conditions, producing the respective amplification products. The terms “influenza A probe(s)” and “influenza B probe(s)” as used herein refer to oligonucleotide probes that specifically anneal to nucleic acid sequences found in the influenza A genome and the influenza B genome, respectively, and enable detection of the respective target amplification products.

The term “5′ to 3′ nuclease activity” refers to an activity of a nucleic acid polymerase, typically associated with the nucleic acid strand synthesis, whereby nucleotides are removed from the 5′ end of a nucleic acid strand and moving toward the 3′ end.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished, as necessary.

The term “extension” or “elongation” when used herein refers to the process by which nucleotides (or other analogous molecules) are incorporated into growing nucleic acid amplification products. For example, a nucleic acid may be extended by a nucleotide incorporating biocatalyst, such as a nucleic acid polymerase, that typically adds nucleotides at the 3′ terminal end of a nucleic acid.

The term “Ct” (or cycle threshold) refers to the number of PCR cycles required for the fluorescent signal to cross a pre-determined fluorescence signal threshold (i.e., when the signal exceeds background level). The term “Amp” (or amplification signal) refers to the calculated average of the final amplitude of the PCR growth curve. It is calculated by dividing the end-point fluorescence by the normalized baseline fluorescence, and thus is a unitless quantity.

The terms “identical” or “identity” in the context of two or more nucleic acid sequences refer to two or more sequences or subsequences that are the same or have a specified percentage of nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated herein by reference.

A “modified nucleotide” refers to an alteration in which at least one nucleotide of an oligonucleotide sequence is replaced by a non-naturally occurring nucleotide that provides a desired property to the oligonucleotide. In some embodiments, modified nucleotides (or “nucleotide analogs”) differ from a natural nucleotide by some modification but still consist of a base or base-like compound, a pentofuranosyl sugar or a pentofuranosyl sugar-like compound, a phosphate portion or phosphate-like portion, or combinations thereof. For example, a “label” may be attached to the base portion of a nucleotide, whereby a “modified nucleotide” is obtained. Exemplary modified nucleotides that can be substituted in the oligonucleotides described herein include, e.g., t-butyl benzyl, C5-methyl-dC, C5-ethyl-dC, C5-methyl-dU, C5-ethyl-dU, 2,6-diaminopurine, C5-propynyl-dC, C5-propynyl-dU, C7-propynyl-dA, C7-propynyl-dG, C5-propargylamino-dC, C5-propargylamino-dU, C7-propargylamino-dA, C7-propargylamino-dG, 7-deaza-2-deoxyxanthosine, pyrazolopyrimidine analog, pseudo-dU, nitro pyrrole, nitro indole, 2′-0-methyl ribo-U, 2′-0-methyl ribo-C, N4-ethyl-dC, N6-methyl-dA, and the like. Many other modified nucleotides that can be substituted in the oligonucleotides are referred to herein or are otherwise known in the art. In certain embodiments, modified nucleotide substitutions modify melting temperatures (Tm) of the oligonucleotides relative to the melting temperatures of corresponding unmodified oligonucleotides. In other embodiments, certain modified nucleotide substitutions can reduce non-specific nucleic acid amplification (e g, minimize primer-dimer formation or the like), and/or increase the yield of an intended target amplicon. Other modified nucleotide substitutions may alter the stability of the oligonucleotide, or provide other desirable features. Examples of these types of nucleic acid modifications are described in, e.g., U.S. Pat. No. 6,001,611, which is incorporated herein by reference. A “modified nucleoside” differs from a natural nucleoside by some modification in the manner as outlined above for a modified nucleotide.

Oligonucleotides including modified oligonucleotides that amplify target nucleic acid molecules can be designed using a variety of computer programs, e.g., a computer program such as OLIGO (Molecular Biology Insights Inc., Cascade, Colo.). Important features when designing oligonucleotides to be used as amplification primers or probes include, but are not limited to, an appropriately-sized amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 8 to 50 nucleotides in length (e.g., 8, 10, 12, 14, 16, 18, 20, 22, 24, 26, 28, 30, 32, 34, 36, 38, 40, 42, 44, 46, 48, or 50 nucleotides in length, or any whole number in between).

SARS-CoV-2 nucleic acids other than those exemplified herein can also be used to detect SARS-CoV-2 in a sample. For example, functional variants can be evaluated for specificity and/or sensitivity by those of skill in the art using routine methods. Representative functional variants can include, e.g., one or more deletions, insertions, and/or substitutions in the SARS-CoV-2 nucleic acids disclosed herein. More specifically, embodiments of the oligonucleotides each include a nucleic acid with a sequence selected from SEQ ID NOs:1-30 or a substantially identical variant thereof in which the variant has at least, e.g., 80%, 90%, or 95% sequence identity to one of SEQ ID NOs: 1-30.

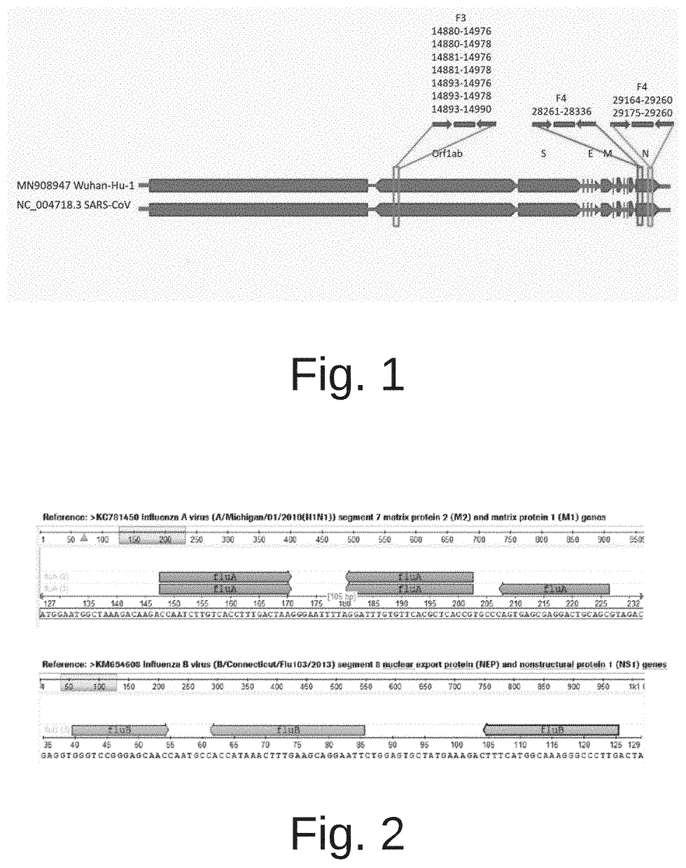

A particular embodiment includes a self-contained nucleic acid analysis tube, which includes a cell lysis zone, a nucleic acid preparation zone, a first-stage amplification zone, a second-stage amplification zone, as shown in FIG. 1 of US Application Publication No. 201000056383, the disclosure of which is incorporated herein by reference. The device may be a flexible device segmented into compartments (or segments) by breakable seals to create a variety of channels and segments of various sizes. The individual compartments may contain various reagents and buffers for processing a sample. Liquid within the tube is moved between blisters by pressure, e.g., pneumatic pressure. In particular, clamps and actuators may be applied to the device in various combinations and with various timings to direct the movement of fluid and to cause the breakable seals to burst. This bursting of the breakable seals may leave an inner device surface that allows fluid flow between the segments. In one embodiment, the flow of the biological sample may be directed toward the distal end of the device as the processing progresses, while the flow of waste may be forced to move in the opposite direction, toward the opening of the device where the sample was initially input. This sample inlet can be sealed, possibly permanently, by a cap with a locking mechanism, and a waste chamber may be located in the cap to receive the waste for storage. A significant benefit of this approach is that the processed sample does not come into contact with surfaces that have been touched by the unprocessed sample. Consequently, trace amounts of reaction inhibitors present in the unprocessed sample that might coat the walls of the device are less likely to contaminate the processed sample.

In exemplary embodiments, one or more reagents can be stored either as dry substance and/or as liquid solutions in device segments. In embodiments where reagents may be stored in dry format, liquid solutions can be stored in adjoining segments to facilitate the reconstitution of the reagent solution. Examples of typical reagents include: lysis reagent, elution buffer, wash buffer, DNase inhibitor, RNase inhibitor, proteinase inhibitor, chelating agent, neutralizing reagent, chaotropic salt solution, detergent, surfactant, anticoagulant, germinant solution, isopropanol, ethanol solution, antibody, nucleic acid probes, peptide nucleic acid probes, and phosphothioate nucleic acid probes. In embodiments where one of the reagents is a chaotropic salt solution, a preferred component is guanidinium isocyanate or guanidinium hydrochloride or a combination thereof. In some embodiments, the order in which reagents may be stored in the device relative to the opening through which a sample is input, reflects the order in which the reagents can be used in methods utilizing the tube. In some embodiments, a reagent includes a substance capable of specifically binding to a preselected component of a sample. For example, a substance may specifically bind to nucleic acid, or a nucleic acid probe may specifically bind to nucleic acids having particular base sequences. In other embodiments, one or more device segments may bear on an inner surface a molecule or substance that binds specifically to a nucleic acid or other component in the reaction mixture, to facilitate isolation or removal of a nucleic acid or other component. See U.S. Pat. No. 10,774,393, the disclosure of which is hereby incorporated by reference in its entirety.

A real-time detection of a signal from a device segment can be achieved by using a sensor, such as a photometer, a spectrometer, a fluorimeter, or a CCD, connected to a block. The format of signal can be an intensity of a light at certain wavelength, such as a fluorescent light, a spectrum, and/or an image, such as image of cells or manmade elements such as quantum dots. For fluorescence detection, an excitation of light from the optical system can be used to illuminate a reaction, and emission light can be detected by the fluorimeter, photometer, spectrometer, or CCD. To detect a plurality of signals having specific wavelengths, different wavelength signals can be detected in series or parallel by dedicated detection channels of a spectrometer.

Embodiments of the present disclosure further provide for kits to detect SARS-CoV-2, or to detect influenza A, influenza B, and SARS-CoV-2 simultaneously. A kit can include primers and probes used to detect the SARS-CoV-2 gene target(s), or to detect the influenza A, influenza B, and SARS-CoV-2 gene targets, together with suitable packaging materials. Representative primers and probes for detection of influenza A, influenza B, and SARS-CoV-2 are capable of hybridizing to target nucleic acid molecules. In addition, the kits may also include suitably packaged reagents and materials needed for DNA immobilization, hybridization, and detection, such solid supports, buffers, enzymes, and DNA standards. Representative examples of primers and probes that amplify and hybridize to SARS-CoV-2 target nucleic acid molecules are provided herein.

Kits can also include one or more fluorescent moieties for labeling the probes or, alternatively, the probes supplied with the kit can be labeled. For example, an article of manufacture may include a donor and/or an acceptor fluorescent moiety for labeling the target probes. Examples of suitable FRET donor fluorescent moieties and corresponding acceptor fluorescent moieties are provided above. Kits can also contain a package insert or package label having instructions thereon for using the SARS-CoV-2 primers and probes to detect SARS-CoV-2 in a sample. Kits may additionally include reagents for carrying out the methods disclosed herein (e.g., buffers, polymerase enzymes, co-factors, or agents to prevent contamination). Kits may also be provided in the form of one or more containers to hold the various components, e.g., primers and/or probes, package inserts, and/or reagents.

### EXAMPLES

The following Examples, Tables, and Figures are provided to aid the understanding of the subject matter. It should be understood that modifications may be made in the disclosures set forth herein without departing from the spirit(s) of the invention.

### Example 1: Selection of candidate primer and probe oligonucleotide sets

An assay to simultaneously detect influenza A, influenza B and SARS-CoV-2 on the Cobas® Liat® Analyzer was developed starting from the Cobas® Liat® Influenza A/B & RSV Assay (Roche Molecular Systems, Pleasanton, Calif.) (“A/B-RSV assay” or “A/B RSV test”), and replacing RSV-specific oligonucleotides with SARS-CoV-2-specific oligonucleotides. The methods and assays described herein to detect SARS-CoV-2 involve targeting two different genes of the SARS-CoV-2 genome—orflab and N gene—to minimize the chance of false negative results due to rise of strains that might be missed if only a single genomic region were targeted. Similarly, an assay to detect SARS-CoV-2 alone on the Cobas® Liat® Analyzer was developed starting from the same A/B-RSV assay, by deleting the oligonucleotides used for detecting influenza A and influenza B, and also replacing RSV detection with SARS-CoV-2 detection. In this version, only oligonucleotides to detect SARS-CoV-2 are present.

A bioinformatics analysis was performed to identify SARS-CoV-2 oligonucleotides that could be multiplexed with oligonucleotides of influenza A and influenza B from the A/B & RSV Assay. The sequences of the influenza A and influenza B primers and probes of the A/B & RSV Assay are shown in Table 1. Influenza A probes are labeled with FAM and BHQ-1; influenza B probes are labeled with Cal Fluor Red 610 (LGC Biosearch Technologies, Novato, Calif.) and BHQ-2. Table 2 shows the primers and probes targeting the orflab region of the SARS-CoV-2 genome described herein. Table 3 shows the primers and probes targeting the N gene of the SARS-CoV-2 genome described herein. The SARS-CoV-2 orflab and N gene probes are labeled with CY5.5 and BHQ-2. FIG. 1 shows the location in the SARS-CoV-2 genome targeted by the various oligonucleotides listed in Tables 2 and 3; FIG. 2 shows the locations targeted by the influenza A and influenza B primers and probes listed in Table 1.

TABLE 1
Influenza A/Influenza B Oligonucleotides

| | SEQ ID | |
|---|---|---|
| Oligonucleotide | NO: | Oligonucleotide Sequence (5′ to 3′) |
| FluA forward primer | 23 | GCTGCAGTCCTCGCTCACT |
| FluA reverse primer | 24 | CCAATCCTGTCACCTCTGACTAA |
| FluA reverse primer | 25 | CCAATCCTGTCACCTCTRACYAA |
| FluA probe | 26 | CGGTGAGCGTGAACACAAATCC |
| FluA probe | 27 | CGGTGAGCGTGAAWACAAACCC |
| FluB forward primer | 28 | CAAGGGCTCTTTGCCATGAAA |
| FluB reverse primer | 29 | GGGTCCGGGAGCAAC |
| FluB probe | 30 | AATTCCTGCTTCAAAGTTTATGGT |

FluA = influenza A; FluB = Influenza B; R = A or G; Y = C or T; W = A or T

TABLE 2
SARS-CoV-2 oligonucleotides: orf1ab

| | SEQ ID | |
|---|---|---|
| Description | NO: | Oligonucleotide Sequence (5′ to 3′) |
| Orf1ab Forward primer | 1 | TGATTGTTACGATGGTGGCTGTA |
| Orf1ab Forward primer | 2 | GATTGTTACGATGGTGGCTGTAT |
| Orf1ab Forward primer | 3 | GGTGGCTGTATTAATGCTAACCAA |
| Orf1ab Reverse primer | 4 | AGCCTTACCCCATTTATTAAATGGAAA |
| Orf1ab Reverse primer | 5 | CTAGCCTTACCCCATTTATTAAATGGA |
| Orf1ab Reverse primer | 6 | TCATAATAAAGTCTAGCCTTACCCCAT |
| Orf1ab Forward primer | 7 | CGTCTGCGGTATGTGGAAA |
| Orf1ab Forward primer | 8 | CTGCGGTATGTGGAAAGGTTA |
| Orf1ab Forward primer | 9 | TCAACTCCGCGAACCCA |
| Orf1ab Reverse primer | 13 | CGCAAACCCGTTTAAAAACGAT |
| Orf1ab Reverse primer | 14 | CACCGCAAACCCGTTTAAAA |
| Orf1ab Reverse primer | 15 | AGACGGGCTGCACTTACA |
| Orf1ab probe | 18 | TCATCGTCAACAACCTAGACAAATCAGCTGGT<PHOS> |
| Orf1ab probe | 19 | AACCCATGCTTCAGTCAGCTGATGCACA<PHOS> |
| Orf1ab probe | 20 | TGCTTCATCAGCTGATGCACAATCGTT<PHOS> |

PHOS = 3′-terminal phosphate group

TABLE 3
SARS-CoV-2 oligonucleotides: N gene

| | SEQ ID | |
|---|---|---|
| Description | NO: | Oligonucleotide Sequence (5′ to 3′) |
| N gene Forward primer | 10 | CGAACAAACTAAAATGTCTGATAATGGA |
| N gene Forward primer | 11 | TTACAAACATTGGCCGCAAAT |
| N gene Forward primer | 12 | GGCCGCAAATTGCACAATTT |
| N gene Reverse primer | 16 | TGAGGGTCCACCAAACGT |
| N gene Reverse primer | 17 | CGTTCCCGAAGGTGTGACT |
| N gene probe | 21 | CAAAATCAGCGAAATGCACCCCGCATT<PHOS> |
| N gene probe | 22 | AGCGCTTCAGCGTTCTTCGGAATGTCG<PHOS> |

PHOS = 3′-terminal phosphate group

### Example 2: Selection of SARS-CoV-2 Oligonucleotide Sets—Orflab

Various candidate oligonucleotide sets directed against the SARS-CoV-2 orflab gene region (Table 2 and Table 4) were evaluated for their performance when used in combination with the oligonucleotides from the A/B-RSV test for detection of influenza A and influenza B. Table 4 shows the oligonucleotide sets and their components. For example, oligonucleotide set F3-Set1 targets a region within the SARS-CoV-2 orflab gene, and includes oligonucleotides of SEQ ID NO:1 (forward primer), SEQ ID NO: 18 (probe), and SEQ ID NO:4 (reverse primer).

TABLE 4
SARS-CoV-2 oligonucleotide sets

| Oligonucleotide | SARS-CoV-2 Target | Oligonucleotide SEQ |
|---|---|---|
| Set Name | Region | ID NOs |
| F3-Set 1 | orf1ab gene | 1\|18\|4 |
| F3-5et 2 | orf1ab gene | 1\|18\|5 |
| F3-Set 3 | orf1ab gene | 2\|18\|4 |
| F3-Set 4 | orf1ab gene | 2\|18\|5 |
| F3-Set 5 | orf1ab gene | 3\|18\|4 |
| F3-Set 6 | orf1ab gene | 3\|18\|5 |
| F3-Set 7 | orf1ab gene | 3\|18\|6 |
| F4-Set 5 | N gene | 10\|21\|16 |
| F4-Set 6 | N gene | 11\|22\|17 |
| F4-Set 7 | N gene | 12\|22\|17 |

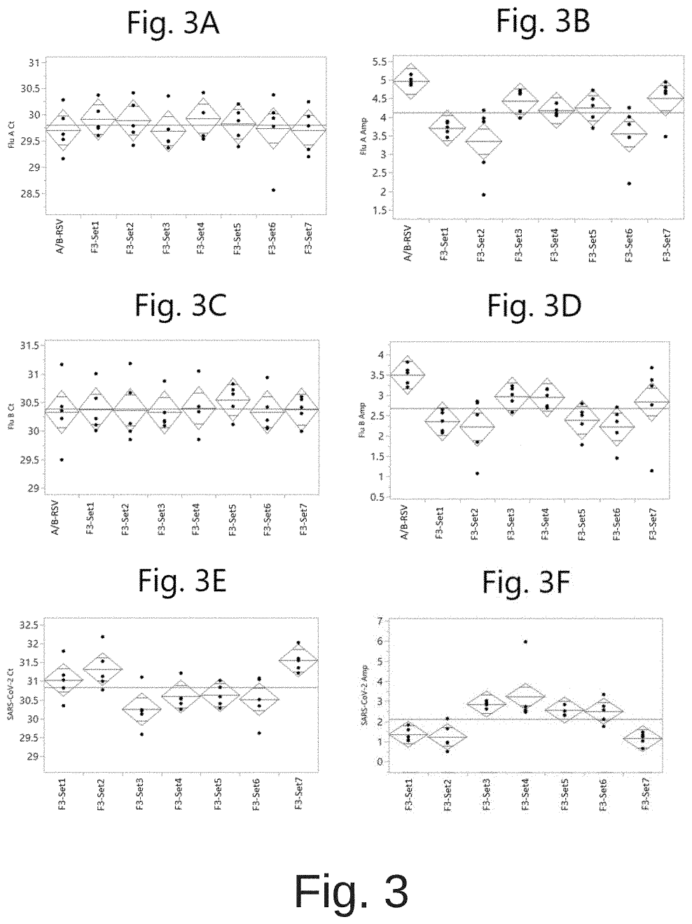

The RSV oligonucleotides used in the A/B & RSV assay were replaced by various SARS-CoV-2 orflab candidate oligonucleotide sets at an identical concentration, and the analyses were performed on the Cobas® Liat® system, using the A/B & RSV assay tube packing configuration, script and calling tool for data analysis. Purified influenza A (Brisbane/59/07) and influenza B (Malaysia/2506/04) RNAs and SARS-CoV-2 nCoV1 transcript (nt14880-15020 from SARS-CoV-2 isolate Wuhan-Hu-1 (GenBank Accession Number MN9089470)) were diluted in MultiPrep Specimen Diluent (Roche Molecular Systems, Branchburg, N.J.) (also known as Bulk Generic Specimen Diluent) and used in assay performance studies. No false positives were observed in three negative runs with each of the seven F3 oligonucleotide sets (F3 sets were those directed to orflab). With nCoV1 RNA transcript at 100 copies/test, oligonucleotide sets F3-Set 3, F3-Set 4, F3-Set 5 and F3-Set 6 showed better performance than the other three oligonucleotide sets (FIGS. 3E and 3F). For influenza A and influenza B, when compared to the results using the A/B & RSV Assay, the Ct was not impacted by multiplexing with these SARS-CoV-2 oligonucleotide sets, whereas only oligonucleotide sets F3-Set 3, F3-Set 4, and F3-Set 5 showed relatively comparable performance to the A/B & RSV Assay (FIGS. 3A-3D). Therefore, F3-Set 3, F3-Set 4, and F3-Set 5 were selected for further performance testing.

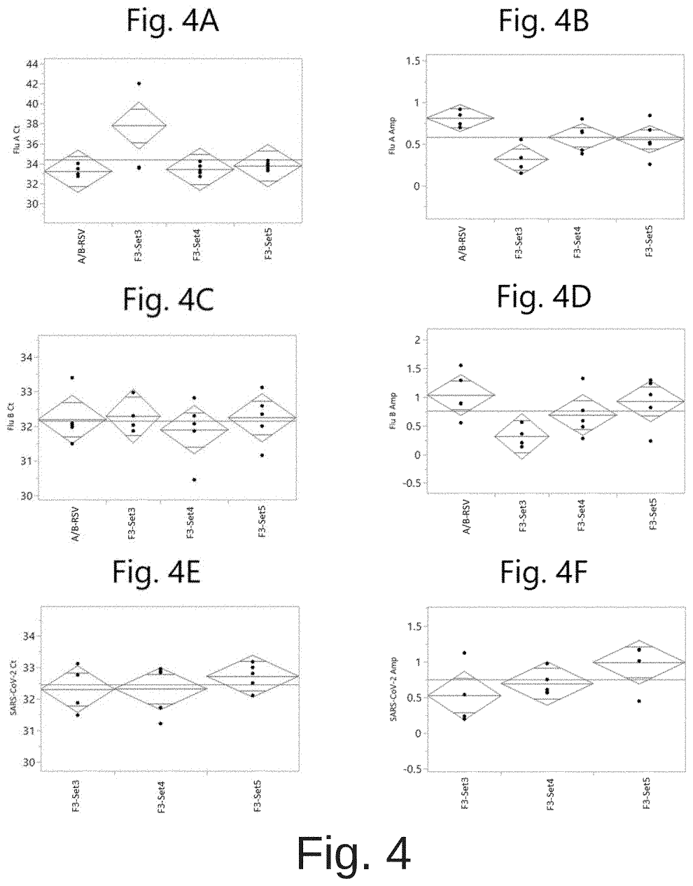

Further performance assessment of the selected SARS-CoV-2 F3 oligonucleotide sets was conducted with 3×LoD influenza A (4.92×10⁻³ TCID₅₀/mL), 3×LoD influenza B (1.67×10⁻³ TCID₅₀/mL) and 400 copies/mL of AccuPlex™ SARS-CoV-2 Verification Panel Member 1 (SeraCare, Milford, Mass., USA) in simulated Universal Transport Media (“sUTM”). Compared to F3-Set 3 and F3-Set 4, F3-Set 5 showed comparable Ct and higher Amp for SARS-CoV-2 detection (FIGS. 4E-F), and better performance for influenza A and influenza B (FIG. 4A-D). It is noted that, by comparison with FluA/B-RSV, F3-Set 5 showed only slightly decreased Amp for influenza A and influenza B. Therefore, F3-Set 5 was selected for further studies as the oligonucleotide set for detecting the SARS-CoV-2 orflab gene.

### Example 3: Selection of SARS-CoV-2 Oligonucleotide Sets—N Gene

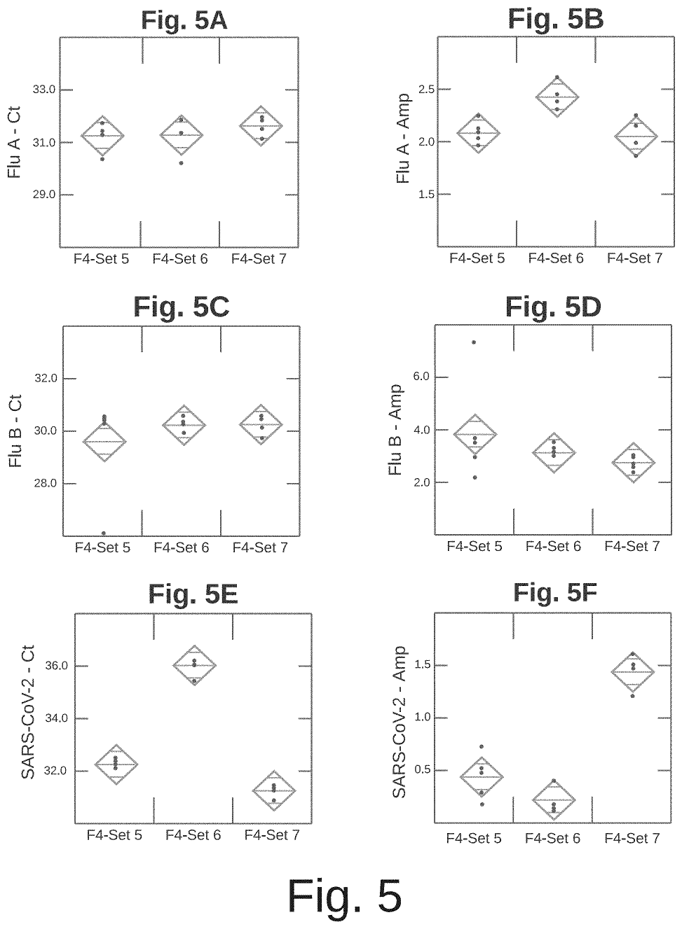

To build a dual target SARS-CoV-2 test, seven sets of oligonucleotides targeting the N gene were prepared using a bioinformatics approach (Table 3 and Table 4). Initial oligonucleotide screening with oligonucleotides targeting the N gene was conducted directly in Cobas® Liat® full tubes containing sUTM. No false positives were observed in four pure negative runs (no sample matrix added) with each of the F4 oligonucleotide sets. For AccuPlex™ Panel Member 1 at 1000 copies/mL, the oligonucleotide set F4-Set 7 showed significantly better performance than other F4 oligonucleotide sets (FIGS. 5E-F). In addition, the oligonucleotide set F4-Set 7 showed acceptable performance in the presence of 10× influenza A (1.64×10⁻² TC1D₅₀/mL) (FIGS. 5A-B) and 10× influenza B (5.58×10⁻³ TC1D₅₀/mL) (FIGS. 5C-D). Therefore, the oligonucleotide set F4-Set 7 was selected for further performance testing.

### Example 4: Single Target and Dual Target Assays Vs. The Cobas® Liat® Influenza A/B & RSV Assay in Detection of Influenza a and Influenza B

Tests were performed to compare performance of the Cobas® Liat® A/B & RSV Assay to that of an influenza A/B & SARS-Cov-2 single target test (“STA”) (orflab target only) and dual target test (“DTA”) (orflab and N gene targets) in detection of influenza A and influenza B. For both single and dual target tests, ten pure negative runs (no sample matrix added) and ten negative runs (with sUTM added) revealed no false positive signals. The performances of the STA with the F3-Set 5 oligo set and the DTA with the F3-Set 5 and F4-Set 7 oligo sets in detecting influenza A and influenza B were assessed in sUTM and UTM. In sUTM, the target inputs were 3×LoD influenza A (4.92×10⁻³ TC1D₅₀/mL), 3×LoD influenza B (1.67×10⁻³ TC1D₅₀/mL) and 400 copies/mL of AccuPlex™ Panel Member 1. In UTM, the target inputs were 3×LoD influenza A (4.92×10⁻³ TC1D₅₀/mL), 3×LoD influenza B (1.67×10⁻³ TC1D₅₀/mL) and 50 copies/mL of AccuPlex™ Reference Material.

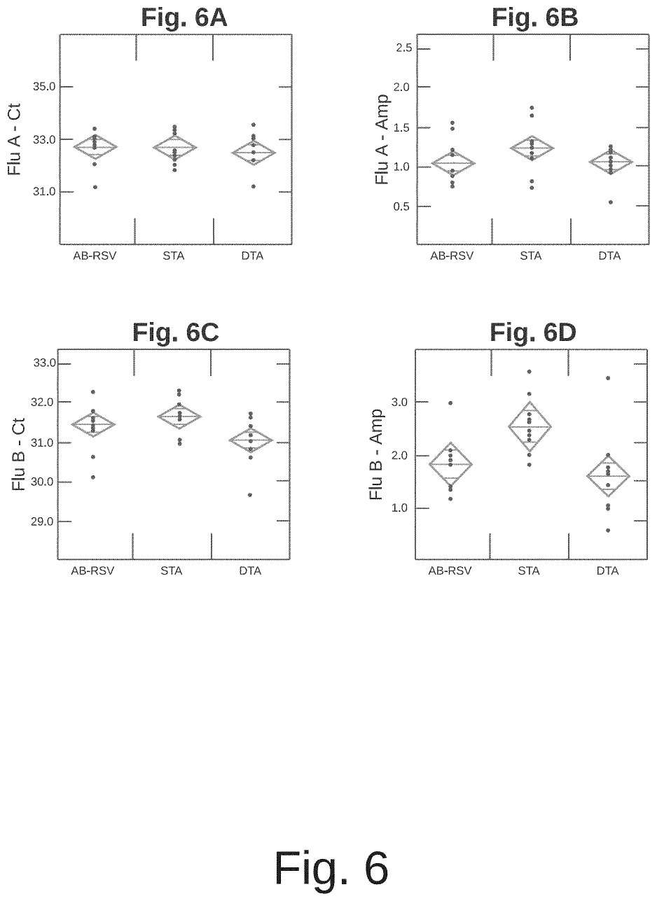

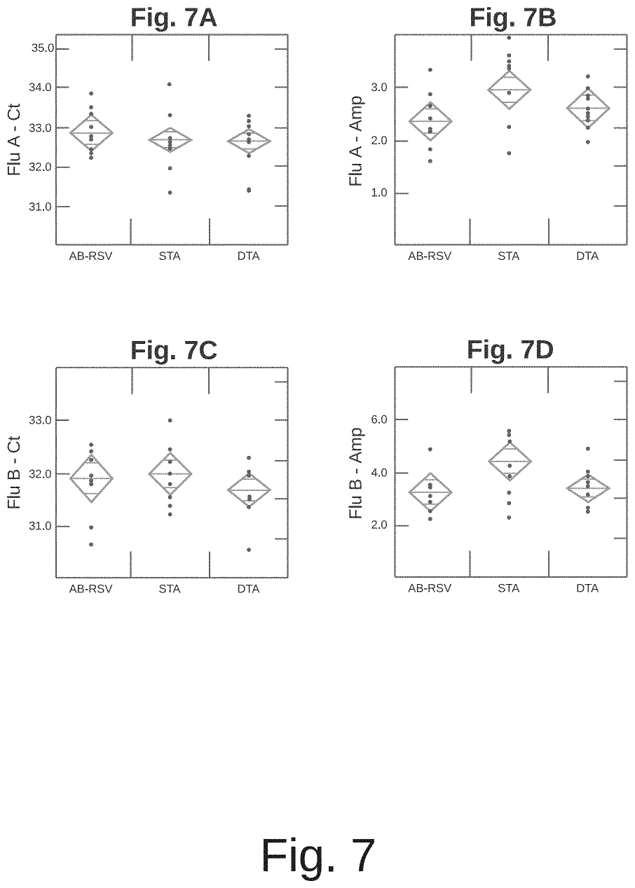

As shown in FIG. 6, the performance of the STA and DTA were comparable to that of the A/B & RSV Assay in detection of influenza A and influenza B in sUTM, as measured in Ct values (FIGS. 6A and 6C). The STA showed higher Amp values for influenza A and influenza B in sUTM, and the DTA showed comparable Amp values for influenza A and influenza B in sUTM (FIGS. 6B and 6D). Similar results were obtained when the targets were assessed in UTM, as seen in FIG. 7. The STA and DTA were comparable to the A/B-RSV Assay in detection of influenza A and influenza B in UTM, as measured in Ct values (FIGS. 7A and 7C). The STA showed higher Amp for influenza A and influenza B in UTM, and the DTA showed comparable Amp for influenza A and influenza B in UTM (FIGS. 7B and 7D).

### Example 5: Single Target and Dual Target Assays Vs. The A/B-RSV Assay in Detection of SARS-CoV-2

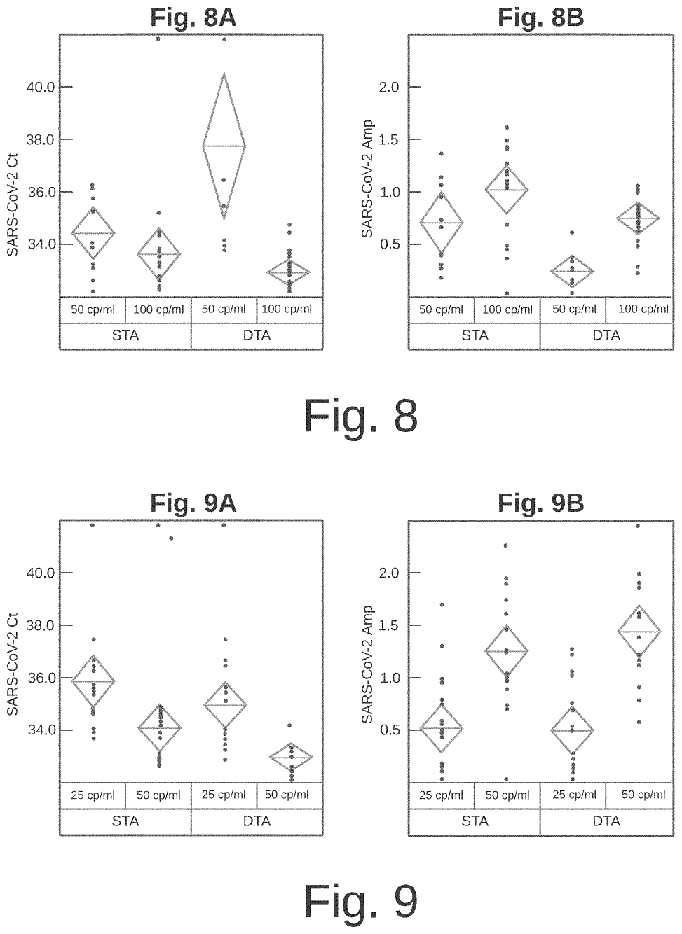

STA and DTA showed SARS-CoV-2 detection sensitivity for AccuPlex™ Panel Member 1 at 100 copies/mL in sUTM (greater than or equal to 95% hit rate) (Table 5). Both tests performed well at both 100 copies/ml and 50 copies/ml, but the DTA had a slightly higher Ct value at 50 copies/ml than the STA (FIG. 8A). For both 100 copies/mL and 50 copies/mL, the STA showed a higher Amp than the DTA (FIG. 8B).

In UTM, both the STA and the DTA showed SARS-CoV-2 detection sensitivity of AccuPlex™ Reference Material, at 50 copies/mL and 25 copies/mL, respectively (95% hit rate) (Table 5). Both tests had slightly higher Ct values at 25 copies/ml than at 50 copies/ml (FIG. 9A). For both STA and DTA, higher Amp values were measured at 50 copies/mL than at 25 copies/mL, as expected, with similar values obtained for both tests (FIG. 9B).

TABLE 5
SARS-CoV-2 STA and DTA performance

| | Hit Rate | | | |
|---|---|---|---|---|
| | Accuplex ™ | | Accuplex ™ | |
| | Member 1 (sUTM) | | Reference Material (UTM) | |
| Assay | 100 copies/mL | 50 copies/mL | 50 copies/mL | 25 copies/mL |
| STA | 95% (19/20) | 100% (10/10) | 95% (19/20) | 90% (18/20) |
| DTA | 100% (20/20) | 60% (6/10) | 100% (15/15) | 95% (19/20) |

### Example 6: SARS-CoV-2 LoD Studies

LoD (Limit of Detection) studies determine the lowest detectable concentration of target sequence at which greater than or equal to 95% of all (true positive) replicates test positive. To determine the LoD for SARS-CoV-2 using the methods described herein, a heat inactivated cultured sample of a SARS-CoV-2 isolate from a US patient (USA-WA1/2020, catalog number 0810587CFHI-0.5 mL, lot number 324047, 3.16×10⁶ TC1D₅₀/mL; ZeptoMetrix, Buffalo, N.Y., USA) was serially diluted in pooled negative clinical nasopharyngeal swab matrix (NNPS). Five concentration levels, with 2-fold serial dilutions between the levels, were tested with 20 replicates per concentration except for the highest concentration level, where only 10 replicates were tested. In addition, three replicates of a blank sample (i.e, pooled NNPS) were tested to ensure the matrix was negative for SARS-CoV-2. For LoD testing, three pilot lots of DTA assay tubes, with approximately equal number of replicates per lot, 20 Cobas® Liat® analyzers (4-5 runs per analyzer) and two independent dilution series (equal number of replicates per dilution series) were used in the study.

As shown in Table 6, the concentration level with observed hit rates greater than or equal to 95% was 0.012 TCID₅₀/mL for SARS-CoV-2. As shown in Table 7, the Probit predicted 95% hit rates were 0.010 TC1D₅₀/mL for SARS-CoV-2.

LoD was also determined using a recombinant SARS-CoV-2 virus AccuPlex™ SARS-CoV-2 Verification Panel, Member 2, at 1×10⁴ copies/mL. The resulting LoD by Probit prediction was 58 copies/mL, with 95% CI of 41-124 (data not shown).

TABLE 6
SARS-CoV-2 LoD

| | Concentration | Total valid | Hit | Mean |
|---|---|---|---|---|
| Strain | [TCID₅₀/mL] | results | rate [%] | Ct* |
| USA-WA1/2020 | 0.048 | 10 | 100 | 32.6 |
| (stock concentration | 0.024 | 20 | 100 | 33.5 |
| 3.16 × 10⁶ TCID₅₀/mL) | 0.012 | 20 | 100 | 35.2 |
| | 0.006 | 20 | 70 | 35.7 |
| | 0.003 | 20 | 25 | 36.7 |

*Calculations only include positive results.

TABLE 7
SARS-CoV-2 LoD: Probit analysis

| Strain | Probit Predicted 95% Hit Rate [TCID₅₀/mL] |
|---|---|
| USA-WA1/2020 | 0.010 |
| (stock concentration | (95% CI: 0.007-0.018) |
| 3.16 × 10⁶ TCID₅₀/mL) | |

### Example 7: Flu A/Flu B LoD Analysis

This study showed that the influenza A and influenza B analytical sensitivity of the dual target assay described herein is equivalent to that of the A/B-RSV Assay.

Cultured influenza A virus strain Brisbane/59/07 (catalog number 0810244CF, lot number 312296, 1.41×105 TCID₅₀/mL; ZeptoMetrix, NY, USA) and influenza B virus strain Florida/04/06 (catalog number 0810255CF, lot number 312479, 1.41×105 TCID₅₀/mL; ZeptoMetrix, NY, USA) were spiked into a pooled negative clinical nasopharyngeal swab matrix (NNPS) and then serially diluted and tested using both the DTA and the A/B-RSV test.

Five concentration levels, with 2-fold serial dilutions between the levels, were tested with a total of 20 replicates per concentration except for the highest concentration level, where only 10 replicates were tested. In addition, three replicates of a blank sample (i.e, pooled NNPS) were tested to ensure the matrix is negative for influenza A and influenza B. Three pilot lots of DTA tubes, 20 Cobas® Liat® analyzers (4-5 runs per analyzer) and 2 independent dilution series (equal number of replicates per dilution series) were used to in the study. One lot of A/B-RSV reagents was used in the study.

The concentration levels with observed hit rates greater than or equal to 95% were 0.001 TCID₅₀/mL for influenza A (Table 8) and 0.004 TCID₅₀/mL for influenza B (Table 9) for both the dual target assay and the A/B-RSV test. One of the influenza B replicates generated a valid “indeterminate” result by the A/B-RSV test; this replicate was included in the total replicates for hit rate calculation, since it is a valid replicate.

As shown in Table 10, the Probit predicted 95% hit rates for influenza A were 0.0007 and 0.0009 TCID₅₀/mL for the dual target assay and the A/B-RSV test, respectively. The Probit predicted 95% hit rates for influenza B were 0.0018 and 0.0026 TCID₅₀/mL for the dual target assay and the A/B-RSV test, respectively. The overlapping confidence intervals demonstrated that the analytical sensitivities of the DTA and the A/B-RSV test are equivalent for influenza A and influenza B detection.

TABLE 8
Influenza A LoD analysis

| | Concentration | # Valid | Hit rate | Hit rate [%]- | mean Ct*- | mean Ct*- |
|---|---|---|---|---|---|---|
| Strain | [TCID₅₀/mL] | results | [%]-DTA | A/B-RSV | DTA | A/B-RSV |
| A/Brisbane/59/07 | 0.008 | 10 | 100% | 100% | 31.2 | 31.9 |
| (stock concentration | 0.004 | 20 | 100% | 100% | 32.4 | 32.8 |
| 1.41 × 10⁵ TCID₅₀/mL) | 0.002 | 20 | 100% | 100% | 33.7 | 33.6 |
| | 0.001 | 20 | 100% | 100% | 34.8 | 34.5 |
| | 0.0005 | 20 | 75% | 50% | 35.4 | 35.5 |

*Calculations only include positive results

TABLE 9
Influenza B LoD analysis

| | Concentration | # Valid | Hit rate | Hit rate [%]- | mean Ct*- | mean Ct- |
|---|---|---|---|---|---|---|
| Strain | [TCID₅₀/mL] | results | [%]-DTA | A/B-RSV | DTA | A/B-RSV |
| B/Florida/04/06 | 0.008 | 10 | 100% | 100% | 30.8 | 31.2 |
| (stock concentration | 0.004 | 20 | 100% | 100% | 31.0 | 31.9 |
| 1.41 × 10⁵ TCID₅₀/mL) | 0.002 | 20 | 90% | 90% | 33.1 | 33.2 |
| | 0.001 | 20 | 95% | 70% | 33.2 | 33.5 |
| | 0.0005 | 20 | 55% | 55% | 33.8 | 34.4 |

*Calculations only include positive results.

TABLE 10
Influenza A/B LoD: Probit analysis

| | Probit Predicted 95% Hit Rate [TCID₅₀/mL] | | | |
|---|---|---|---|---|
| | Influenza A | | Influenza B | |
| Strain | DTA | FluA/B-RSV | DTA | FluA/B-RSV |
| A/Brisbane/59/07 | 0.0007 | 0.0009 | 0.0018 | 0.0026 |
| B/Florida/04/06 | (95% CI: 0.0006- | (95% CI: 0.0007- | (95% CI: 0.0012- | (95% CI: 0.0017- |
| | na*) | 0.0029) | 0.0062) | 0.0087) |

*Calculations only include positive results.
DTA = dual target assay

Additional analysis of LoD was done using maximum likelihood method (MLE) for tighter confidence intervals (since Probit analysis did not have a confidence interval for influenza A). The 83% CI was shown to be overlapping for influenza A (0.0007-0.0012 for the DTA and 0.0010-0.0017 for A/B-RSV test) and influenza B (0.0014-0.0022 for the DTA and 0.0018-0.0029 for A/B-RSV test), demonstrating no statistically significant difference between the dual target assay and the A/B-RSV test.

### Example 8: Detection of Influenza A/B in Clinical Samples

Archived influenza A and influenza B positive clinical samples were tested using the dual target assay and A/B-RSV test. The clinical samples were nasopharyngeal swab specimens in UTM, and were acquired from various external vendors. In addition, the CDC 2019 Human Influenza Virus Panel, which includes influenza A (H1N1) strain Brisbane/02/2018, influenza A (H3N2) strain Perth/16/2009; influenza B Victoria lineage Colorado/06/2017 and influenza B Yamagata lineage Phuket/3073/2013, was diluted into NNPS and tested using both the A/B-RSV test and the dual target test.

As shown in Tables 11-13, all positive clinical samples tested positive by both tests. Referring to Table 11, both tests detected 100% of the influenza A samples, and did not report a false positive influenza B result. Similarly, as shown in Table 12, both tests detected 100% of the influenza B samples, and did not report a false positive influenza A result. Lastly, Table 13 shows that the two influenza A and two influenza B strains/lineages of the CDC 2019 Human Influenza Virus Panel were properly detected by both the dual target assay and the A/B-RSV test.

TABLE 11
detection of influenza A

| | | | | | | Internal |
|---|---|---|---|---|---|---|
| | | Influenza A | | Influenza B | | Control |
| Sample ID | Test | Ct | Result | Ct | Result | Ct |
| FluA-103- | A/B-RSV | 22.2 | Detected | 42.0 | Not Detected | 31.5 |
| R-NP-PCR | DTA | 21.7 | Detected | 42.0 | Not Detected | 29.8 |
| FluA-131- | A/B-RSV | 29.3 | Detected | 42.0 | Not Detected | 30.5 |
| R-NP-PCR | DTA | 28.8 | Detected | 42.0 | Not Detected | 30.5 |
| FluA-I35- | A/B-RSV | 31.9 | Detected | 42.0 | Not Detected | 32.3 |
| R-NP-PCR | DTA | 31.6 | Detected | 42.0 | Not Detected | 32.9 |
| FluA-137- | A/B-RSV | 23.4 | Detected | 42.0 | Not Detected | 29.5 |
| R-NP-PCR | DTA | 22.9 | Detected | 42.0 | Not Detected | 29.2 |
| FluA-138- | A/B-RSV | 33.0 | Detected | 42.0 | Not Detected | 29.9 |
| L-NP-PCR | DTA | 32.5 | Detected | 42.0 | Not Detected | 30.0 |

Note:
Ct of 42 indicates negative results

TABLE 12
detection of influenza B

| | | | | | | Internal |
|---|---|---|---|---|---|---|
| | | Influenza A | | Influenza B | | Control |
| Sample ID | Test | Ct | Result | Ct | Result | Ct |
| T000167- | A/B-RSV | 42.0 | Not Detected | 17.8 | Detected | 32.9 |
| 4310001 | DTA | 42.0 | Not Detected | 17.5 | Detected | 30.6 |
| T000167- | A/B-RSV | 42.0 | Not Detected | 17.4 | Detected | 32.5 |
| 4310201 | DTA | 42.0 | Not Detected | 17.3 | Detected | 30.2 |
| T000167- | A/B-RSV | 42.0 | Not Detected | 22.6 | Detected | 32.1 |
| 4310301 | DTA | 42.0 | Not Detected | 26.7 | Detected | 29.3 |
| T000167- | A/B-RSV | 42.0 | Not Detected | 18.5 | Detected | 32.7 |
| 4310601 | DTA | 42.0 | Not Detected | 18.5 | Detected | 31.0 |
| 602_NPS_ | A/B-RSV | 42.0 | Not Detected | 25.3 | Detected | 31.0 |
| L_PLS | DTA | 42.0 | Not Detected | 24.4 | Detected | 30.6 |
| 817_NPS_ | A/B-RSV | 42.0 | Not Detected | 36.0 | Detected | 29.6 |
| R_PLS | DTA | 42.0 | Not Detected | 35.3 | Detected | 29.3 |

Note:
Ct of 42 indicates negative results

TABLE 13
detection of strains in CDC 2019 Human Influenza Virus Panel

| | | Titer | | | | | | Internal |
|---|---|---|---|---|---|---|---|---|
| Strain | Subtype/ | (EID₅₀/ | | Influenza A | | Influenza B | | Control |
| Name | Lineage | mL) | Test | Ct | Result | Ct | Result | Ct |
| A/Brisbane/ | Influenza A/ | 20 | A/B-RSV | 30.5 | Detected | 42 | Not Detected | 29.7 |
| 02/2018 | H1N1pdm09 | | DTA | 31.4 | Detected | 42 | Not Detected | 31.1 |
| A/Perth/16/ | Influenza A/ | 20 | A/B-RSV | 31.9 | Detected | 42 | Not Detected | 30.9 |
| 2009 | H3N2 | | DTA | 31.7 | Detected | 42 | Not Detected | 31.2 |
| B/Colorado/ | Victoria | 20 | A/B-RSV | 42 | Not Detected | 28.4 | Detected | 33.5 |
| 06/2017 | | | DTA | 42 | Not Detected | 28.3 | Detected | 30.8 |
| B/Phuket/ | Yamagata | 40 | A/B-RSV | 42 | Not Detected | 33.9 | Detected | 32.7 |
| 3073/2013 | | | DTA | 42 | Not Detected | 32.5 | Detected | 30.6 |

Note:
Ct of 42 indicates negative results

### Example 9: Cross-Reactivity of DTA with SARS-CoV-1

Cross-reactivity of the dual target assay with SARS-CoV-1 was evaluated by testing inactivated SARS-CoV-1 whole virus. Gamma irradiated cultured SARS-CoV-1, Urbani strain (catalog number NR-9547, lot number 58542036, BEI Resources, VA, USA) was serially diluted into pooled negative nasopharyngeal swab samples in UTM to final concentrations between 1.0×105-1.0×101 pfu/mL. The results are shown in Table 14. None of the concentrations tested interfered with the dual target assay performance by generating false positive results.

TABLE 14
SARS-CoV-1 cross-reactivity detection

| | DTA | | | |
|---|---|---|---|---|
| SARS-CoV-1 | SARS-CoV-2 | FluA | FluB | IC |
| Concentration | Result | Result | Result | Ct |
| 0 | Not Detected | Not Detected | Not Detected | 30.6 |
| 1.00 × 10⁵ pfu/mL | Not Detected | Not Detected | Not Detected | 31.6 |
| 1.00 × 10⁴ pfu/mL | Not Detected | Not Detected | Not Detected | 32.4 |
| 1.00 × 10³ pfu/mL | Not Detected | Not Detected | Not Detected | 30.6 |
| 1.00 × 10² pfu/mL | Not Detected | Not Detected | Not Detected | 30.9 |
| 1.00 × 10¹ pfu/mL | Not Detected | Not Detected | Not Detected | 31.3 |

### Example 10: Clinical Performance of DTA

The clinical performance of the dual target test was evaluated using 56 known SARS-CoV-2 positive nasopharyngeal clinical samples and 231 negative clinical samples (a mixture of nasopharyngeal and nasal swab samples) collected in UTM from patients with a suspected respiratory infection. Testing of clinical samples was performed with the dual target test, and performance compared with that of the commercially available Cobas® SARS-CoV-2 test for use on the Cobas® 6800/8800 Systems (Roche Molecular Systems, Pleasanton, Calif.).

Known SARS-CoV-2 positive specimens were acquired from BioCollections Worldwide, Inc. (Oakland, Calif.) and from UC Davis (Davis, Calif.). All positive samples were nasopharyngeal swabs collected in UTM. Negative clinical specimens used in the study were collected in the US prior to the SARS-CoV-2 pandemic from individuals suspected of an upper respiratory infection. These negative specimens include both nasal and nasopharyngeal swab specimens collected in UTM using mini-tip flocked swabs and regular flocked swabs. Clinical specimens were collected by qualified personnel according to the package insert of the collection device. Samples were handled as described in the package insert of the collection device and stored frozen until use.

A total of 56 known SARS-CoV-2 positive nasopharyngeal swab specimens and 231 SARS-CoV-2 negative specimens (a mixture of nasopharyngeal swab and nasal swab specimens) were tested. As shown in Table 15, all 56 SARS-CoV-2 positive samples tested positive with both the DTA and the Cobas® 6800/8800 SARS-CoV-2 Test. Of the 231 negative specimens tested, five generated invalid results and upon retest, two remained invalid for the dual target test. The initial and after retest invalid rates in this study for the dual target test were 1.7% (5/287) and 0.7% (2/287) respectively. The negative samples that remained invalid were excluded; thus, 229/231 negative samples were included. One negative sample tested positive for influenza A, and this result was confirmed with an A/B-RSV test (data not shown). All 229 specimens tested negative for SARS-CoV-2 using both the DTA and the Cobas® 6800/8800 SARS-CoV-2 test, and were used for correlation analysis (Table 16). The results demonstrated 100% positive percent agreement (PPA) and 100% negative percent agreement (NPA) between the two tests.

TABLE 15
SARS-CoV-2 clinical performance

| | | | | | cobas ^{®} SARS-CoV-2 Test on | | | |
|---|---|---|---|---|---|---|---|---|
| | | | | | cobas ^{®} 6800/8800 System | | | |
| | | Dual Target test | | | nCoV (Target 1) | | SARB-1 (Target 2) | |
| Clinical Specimen | N | Positive | Negative | Invalid | Positive | Negative | Positive | Negative |
| SARS-CoV2 positive | 56 | 56 | 0 | 0 | 56 | 0 | 56 | 0 |
| SARS-CoV2 negative | 231 | 0 | 229 | 2 | 0 | 231 | 0 | 231 |

TABLE 16
SARS-CoV-2 correlation analysis

| | | cobas ® SARS-CoV-2 on | |
|---|---|---|---|
| | | cobas ® 6800/8800 Systems | |
| | | positive | negative |
| Dual Target Assay | positive | 56 | 0 |
| | negative | 0 | 229* |

| | |
|---|---|
| PPA | 100% (95% CI: 93.6%-100%) |
| NPA | 100% (95% CI: 98.4%-100%) |

*2 repeated invalid samples were not included in the correlation analysis

### Example 11: Flu A/Flu B/SARS-CoV-2 Competitive Inhibition (Co-Infection)

Competitive inhibition among influenza A, influenza B, and SARS-CoV-2 was evaluated by performing a series of dilution experiments using co-infected samples that contained one panel target at high concentration and one or more additional panel targets at low concentrations. The purpose of these experiments was to identify concentrations at which the presence of the high concentration target would inhibit detection of the low concentration target(s) due to competition. Low concentrations were defined as ˜3×LoD. High concentration targets were defined as either high (Ct 20-24) or very high (Ct 12-16) titers. Samples were tested in a series of dilutions until the low concentration targets were detected at 100% hit rate.

Inactivated SARS-CoV-2 (USA-WA1/2020), cultured influenza A (Brisbane/59/07) virus, and cultured influenza B (Florida/04/06 and Colorado/06/2017) viruses were prepared in pooled negative nasopharyngeal swabs eluted in UTM sample matrix. Three replicates were tested per condition. The concentrations tested in the dilution experiments are presented in both ID50/mL and copies/mL.

The concentration of each viral stock in copies/mL was quantified using a RT-ddPCR (reverse transcriptase-droplet digital PCR) assay in a single target, single-plex assay with target specific PCR primers and probe sets designed to independently amplify influenza A, influenza B, or SARS-CoV-2 using the One-Step RT-ddPCR Advanced Kit for Probes (Bio-Rad, cat #1864021).

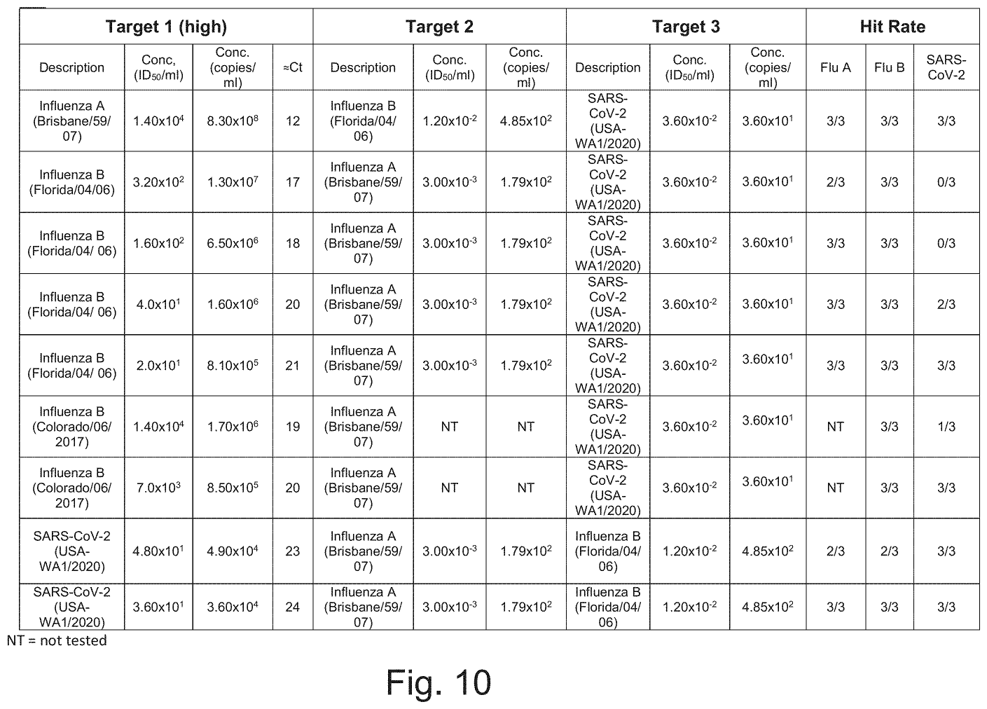

A summary of the testing results is shown in FIG. 10. Influenza A high target samples exhibited an average Ct of 12, while the influenza B and SARS-CoV-2 target samples yielded an average Ct between 20-24. The low target concentrations (Target 2 and 3) were ˜3×LoD.

Results of the study showed that influenza B required concentrations above 8.10×105 copies/mL to cause inhibition of SARS-CoV-2 detection at low concentration. SARS-CoV-2 required concentrations above 3.60×104 copies/mL to cause inhibition of both influenza A and influenza B detection at low concentrations.

### Example 12: Interfering Microorganisms

Interfering microorganism study evaluates whether non-influenza microorganisms that may be present in nasopharyngeal swab samples can interfere in the detection of influenza A or influenza B. The panel comprising human genomic DNA and 35 microorganisms tested in the cross-reactivity study was tested for potential interference. Bacteria and Candida albicans were tested at ≥10⁶ CFU/mL and viruses were tested at ≥10⁵ TC1D₅₀/mL or the highest available concentration, in the presence of one influenza A strain and one influenza B strain at ˜3×LoD concentration in negative NPS in UTM matrix. Results show that the presence of human genomic DNA or the microorganisms at the concentrations tested did not interfere with the detection of influenza A or influenza B (Table 17).

TABLE 17
Influenza A/B interfering microorganisms study

| | Test Concentration | Influenza A & Influenza B at ~3× LoD | |
|---|---|---|---|
| Microorganism | (TCID₅₀/mL) | influenza A Result | influenza B Result |
| Adenovirus Type 1 | 9.0 × 10⁵ | + | + |
| Adenovirus Type 7 | 1.4 × 10⁵ | + | + |
| Cytomegalovirus | 4.5 × 10⁴ | + | + |
| Epstein Barr Virus | 2.5 × 10⁵ | + | + |
| Herpes Simplex Virus | 1.4 × 10⁵ | + | + |
| Human Coronavirus 229E | 8.0 × 10³ | + | + |
| Human Coronavirus OC43 | 8.0 × 10⁴ | + | + |
| Human Enterovirus 68 | 1.0 × 10⁵ | + | + |
| Human Metapneumovirus | 7.0 × 10³ | + | + |
| Human Parainfluenza Type 1 | 3.7 × 10⁵ | + | + |
| Human Parainfluenza Type 2 | 7.5 × 10⁵ | + | + |
| Human Parainfluenza Type 3 | 4.5 × 10⁵ | + | + |
| Human Rhinovirus Type 1A | 8.0 × 10⁵ | + | + |
| Measles | 8.0 × 10⁴ | + | + |
| Mumps Virus | 8.0 × 10⁴ | + | + |
| Varicella-Zoster Virus | 4.4 × 10³ | + | + |
| Chlamydia pneumonzae | 8.0 × 10⁴ | + | + |
| | Test Concentration | | |
| Microorganism | (CFU/mL) | influenza A Result | influenza B Result |
| Bordetella pertussis | 2.2 × 10⁶ | + | + |
| Candida albicans | 4.2 × 10⁶ | + | + |
| Corynebacterium sp | 3.6 × 10⁶ | + | + |
| Escherichia coli | 1.9 × 10⁶ | + | + |
| Haemophilus influenzae | 2.3 × 10⁶ | + | + |
| Lactobacillus sp | 1.9 × 10⁶ | + | + |
| Legionella pneumophila | 6.7 × 10⁶ | + | + |
| Moraxella catarrhalis | 2.5 × 10⁶ | + | + |
| Neisseria elongata | 2.0 × 10⁶ | + | + |
| Neisseria meningitidis | 2.2 × 10⁶ | + | + |
| Pseudomonas aeruginosa | 2.3 × 10⁶ | + | + |
| Staphylococcus aureus | 2.4 × 10⁶ | + | + |
| Staphylococcus epidermidis | 1.9 × 10⁶ | + | + |
| Streptococcus pneumoniae | 1.8 × 10⁶ | + | + |
| Streptococcus pyogenes | 2.5 × 10⁶ | + | + |
| Streptococcus salivarius | 4.3 × 10⁶ | + | + |
| | Test Concentration | | |
| Microorganism | (copies/mL) | influenza A Result | influenza B Result |
| Human Genomic DNA | 1.0 × 10⁴ | + | + |
| Mycobacterium tuberculosis † | 2.8 × 10⁶ | + | + |
| Mycoplasma pneumoniae † | 2.9 × 10⁶ | + | + |

† Testing was performed with genomic DNA due to difficulties in propagation of these bacteria

### Example 13—Clinical Studies—Influenza A/B

The clinical performance of the DTA in detecting influenza A and influenza B was evaluated at 12 CLIA-waived healthcare facilities. Prospective nasopharyngeal swab (NPS) specimens were collected from patients with signs and symptoms of respiratory infection in the US during the 2013-2014 and 2014-2015 flu seasons, and were tested prospectively at the study sites. Additionally, retrospective NPS specimens were obtained from two reference laboratories and were distributed to and tested at three of the 12 sites. The retrospective specimens were worked into the daily workload of those sites for testing.

Each patient's specimen was tested for influenza A and influenza B, using the DTA and an FDA-cleared laboratory-based multiplexed real-time reverse transcriptase PCR (RT-PCR) test (comparator test). The results for DTA in detection of influenza A and influenza B were compared against the results from the comparator test. A total of 1,350 prospective NPS specimens and 292 retrospective NPS specimens were included in the performance analysis.

A total of 1,350 prospective nasopharyngeal swab (NPS) specimens were included in the performance analysis (Table 18 and Table 19). Compared to the comparator test, the assay demonstrated positive agreement of 98.3% and 95.2% for influenza A and influenza B, respectively; and negative agreement of 96.0% and 99.4% for influenza A and influenza B, respectively.

TABLE 18
Clinical performance: prospective NPS specimens-Influenza A

| | | Comparator Test | | |
|---|---|---|---|---|
| | | Positive | Negative | Total |
| DTA | Positive | 172 | 47 | 219 |
| | Negative | 3 | 1128 | 1131 |
| | Total | 175 | 1175 | 1350 |

| | % | 95% CI |
|---|---|---|
| Positive agreement | 98.3 | 95.1-99.4 |
| Negative Agreement | 96.0 | 94.7-97.0 |

TABLE 19
Clinical performance: prospective NPS specimens-Influenza B

| | | Comparator Test | | |
|---|---|---|---|---|
| | | Positive | Negative | Total |
| DTA | Positive | 40 | 8 | 48 |
| | Negative | 2 | 1300 | 1302 |
| | Total | 42 | 1308 | 1350 |

| | % | 95% CI |
|---|---|---|
| Positive Agreement | 95.2 | 84.2-98.7 |
| Negative Agreement | 99.4 | 98.8-99.7 |

For retrospective specimen analysis, a total of 292 retrospective nasopharyngeal swab (NPS) specimens were included in the performance analysis (Table 20 and Table 21). Compared to the comparator test, influenza A and influenza B demonstrated positive agreement of 98.7% and 99.0%, respectively; and negative agreement of 99.1% and 99.5% for influenza A and influenza B, respectively.

TABLE 20
Clinical performance: retrospective NPS specimens-Influenza A

| | | Comparator Test | | |
|---|---|---|---|---|
| | | Positive | Negative | Total |
| DTA | Positive | 76 | 2 | 78 |
| | Negative | 1 | 213 | 214 |
| | Total | 77 | 215 | 292 |

| | % | 95% CI |
|---|---|---|
| Positive Agreement | 98.7 | 93.0-99.8 |
| Negative Agreement | 99.1 | 96.7-99.7 |

TABLE 21
Clinical performance: retrospective NPS specimens-Influenza B

| | | Comparator Test | | |
|---|---|---|---|---|
| | | Positive | Negative | Total |
| DTA | Positive | 97 | 1 | 98 |
| | Negative | 1 | 193 | 194 |
| | Total | 98 | 194 | 292 |

| | % | 95% CI |
|---|---|---|
| Positive Agreement | 95.2 | 94.4-99.8 |
| Negative Agreement | 99.4 | 97.1-99.9 |

### Example 14: SARS-CoV-2 LoD Studies

The dual target assay was further modified by removal of the primers and probes used to detect influenza A and influenza B, creating an assay having only primers and probes suitable for detection of SARS-CoV-2 (herein denoted “DT(−) assay”). Preliminary LoD for DT(−) assay was determined using heat-inactivated SARS-CoV2 virus (USAWA1/2020, ZeptoMetrix, Catalog Number 0810587CFHI-0.5 mL). The four-level panel was prepared by spiking SARS-CoV-2 culture into pooled NNPS clinical background to achieve the indicated concentrations. The same panel was also tested against the DTA as a comparator test (Table 22). Twenty replicates of each level were tested using both tests. Results of the LoDs by 95% hit rate and Probit estimates including 95% confidence limits are summarized for both tests.

The DTA(−) assay LoD is 0.012 TC1D₅₀/mL as determined by both 95% hit rate and Probit estimation (Table 22). The comparison to DTA is also shown in Table 22. Although the DT(−) assay showed slightly better analytical sensitivity in this study than the DTA, the overlapping confidence intervals of Probit LoDs indicate equivalent analytical sensitivities between the DT(−) assay and DTA assays for SARS-COV-2 detection.

TABLE 22
DT (-) assay LoD

| | SARS-CoV-2 | DTA | | | | DT (-) assay | | | |
|---|---|---|---|---|---|---|---|---|---|
| | Concentration | | | | Hit Rate | | | | Hit Rate |
| Level | (TCID₅₀/mL) | Avg Ct | Hits | Trials | (%) | Avg Ct | Hits | Trials | (%) |
| 1 | 0.024 | 33.8 | 20 | 20 | 100 | 33.5 | 20 | 20 | 100 |
| 2 | 0.012 | 35.0 | 17 | 20 | 85 | 34.8 | 19 | 20 | 95 |
| 3 | 0.006 | 35.1 | 11 | 20 | 55 | 35.8 | 14 | 20 | 70 |
| 4 | 0.003 | 36.0 | 8 | 20 | 40 | 36.1 | 11 | 20 | 55 |

| | | |
|---|---|---|
| 95% Hit Rate LoD | 0.024 | 0.012 |
| Probit LoD (95% Cis) | 0.016 (0.012-0.029) | 0.012 (0.009-0.027) |

NOTE:
Mean Ct does not include Ct values of 42.0

The data set was also analyzed for Ct and AMP performance between the DT(−) assay and the DTA tests. Mean Ct and mean AMP summaries are shown in Table 23. Although there was no significant difference in mean Ct between the DT(−) assay and DTA tests, there was a notable increase in mean AMPs for DT(−) assay (Table 23). The increase in AMP suggests increased robustness of DT(−) assay over DTA.

TABLE 23
Mean Ct and Amp comparisons for DT (-) assay and DTA

| SARS-CoV-2 | | | | | | | | | | DT (-) assay/ |
|---|---|---|---|---|---|---|---|---|---|---|
| Concentration | DT (-) | | DTA | | | DT (-) | | DTA | | DTA |
| (TCID₅₀/mL) | Mean Ct | N | Mean Ct | N | ΔCt | Mean Amp | N | Mean Amp | N | (mean AMP) |
| 0.024 | 33.5 | 20 | 33.8 | 20 | −0.3 | 1.53 | 20 | 0.60 | 20 | 255% |
| 0.012 | 34.8 | 19 | 35.0 | 17 | −0.2 | 0.72 | 20 | 0.33 | 20 | 218% |
| 0.006 | 35.8 | 14 | 35.1 | 11 | 0.7 | 0.36 | 20 | 0.29 | 19 | 122% |
| 0.003 | 36.1 | 11 | 36.0 | 8 | 0.1 | 0.29 | 20 | 0.22 | 20 | 145% |

NOTE:
Mean Ct does not include Ct values of ≥42.0

### Example 15: DT(−) Assay Inclusivity Study

In silico analysis was performed to determine the predicted ability of the oligonucleotide sets for the DT(−) assay (taxonomy ID 2697049) to detect all SARS-CoV-2 sequences in NCBI (>86K), and in GISAID (>657K) databases (as of 15 Mar. 2021). The predicted impact of each sequence in the two databases to be detected by the oligonucleotide sets was evaluated using an internal sequence comparison software and quantitated as the predicted increase in Ct or probe melting temperature (Tm). A sequence is predicted potentially not being detected by a primer/probe test set if the predicted Ct increases by greater than 5 cycles or the probe Tm is less than 65° C. The mismatch analysis and predicted assay impact for the DT(−) assay oligonucleotide sets (which are identical to the SARS-CoV-2-specific oligonucleotide sets of the DTA) are summarized in Table 24. All sequences in the NCBI and the GISAID databases are predicted to be detected by at least one of the oligonucleotide sets of the DT(−) assay.

TABLE 24
in silico inclusivity analysis of DT(-) assay

| Target | orf1ab | | | | N gene | | | | orf1ab + N gene | | | |
|---|---|---|---|---|---|---|---|---|---|---|---|---|
| Database | NCBI | | GSAID | | NCBI | | GSAID | | NCBI | | GSAID | |
| Total | 86803 | 100% | 658450 | 100% | 86803 | 100% | 658450 | 100% | 86803 | 100% | 658450 | 100% |
| w/mismatch | 791 | 0.91% | 4789 | 0.73% | 2612 | 3.01% | 21021 | 3.19% | 55 | 0.06% | 197 | 0.03% |
| ΔCt >5 or | 0 | 0.00% | 6 | 0.00% | 2 | 0.00% | 41 | 0.00% | 0 | 0.00% | 0 | 0.00% |
| Tm <65° C. | | | | | | | | | | | | |

### Example 16: SARS-CoV-2 Clinical Study—Asymptomatic Samples

The clinical performance of the DT(−) assay was evaluated using a total of 207 nasopharyngeal clinical samples collected in saline from asymptomatic individuals presenting to a single testing facility for COVID-19 screening. Testing of clinical samples was performed with the DT(−) and a highly sensitive FDA-cleared EUA molecular assay that has been approved for COVID-19 screening. As shown in Table 25, the results demonstrated 100% positive agreement with lower bound of the two-sided 95% confidence interval of 84.5%; 98.9% negative agreement with lower bound of the two-sided 95% confidence interval of 96.2% against the comparator method.

TABLE 25
DT(−) assay performance with asymptomatic patient samples
N = 207

| Comparator | DT(−) assay | N |
|---|---|---|
| − | − | 184 |
| + | − | 0 |
| − | + | 2 |
| + | + | 21 |

### Example 17: SARS-CoV-2 Clinical Study—Suspected Positive Samples

The clinical performance of Cobas® SARS-CoV-2 test for the detection of SARS-CoV-2 was evaluated using a total of 230 nasopharyngeal clinical samples collected in UTM from individuals suspected of having a COVID-19 infection, including those with signs and symptoms of a respiratory infection. Testing of clinical samples was performed with Cobas® SARS-CoV-2 test and a highly sensitive FDA-cleared EUA molecular assay that has been approved for diagnostic testing of COVID-19. As shown in Table 26, the results demonstrated 96.1% positive percent agreement (PPA) and 96.8% negative percent agreement (NPA) between the Cobas® SARS-CoV-2 test on the Cobas® Liat® System and the comparator method. All eight discordant specimens (five positives by the Cobas® SARS-CoV-2 test and three positives by the comparator method) were very low positive specimens at or below the limit of detection for the respective assay yielding a positive result.

TABLE 26
DT(-) assay performance with symptomatic patient samples

| | | Comparator assay | |
|---|---|---|---|
| | | positive | negative |
| DT(-) | positive | 73 | 5 |
| assay | negative | 3 | 149 |

| | % | 95% CI |
|---|---|---|
| PPA | 98.7 | 93.0-99.8 |
| NPA | 99.1 | 96.7-99.7 |

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes.

What is claimed is:

1. A method of detecting Severe Acute Respiratory Syndrome Coronavirus 2 (SARS-CoV-2) nucleic acid in a sample, the method comprising:
a) contacting the sample with at least a first set of primers and a second set of primers under conditions suitable for producing one or more amplification products if a target nucleic acid is present in the sample;
b) contacting the sample with at least a first detectable probe and a second detectable probe under conditions suitable for producing a signal from at least one of the first and second detectable probes if one or more amplification products are present; and
c) detecting the signal produced in step b), wherein the presence of the one or more amplification products is indicative of the presence of SARS-CoV-2 nucleic acids in the sample and wherein the absence of the one or more amplification products is indicative of the absence of SARS-CoV-2 nucleic acids in the sample;
wherein the first set of primers comprises a first primer having an oligonucleotide sequence selected from the group consisting of SEQ ID NOs:1-3 and SEQ ID NOs: 7-9, and a second primer having an oligonucleotide sequence selected from the group consisting of SEQ ID NOs:4-6 and SEQ ID NOs: 13-15; and
wherein the second set of primers comprises a third primer having an oligonucleotide sequence selected from the group consisting of SEQ ID NOs: 10-12, and a fourth primer having an oligonucleotide sequence selected from the group consisting of SEQ ID NOs:16-17; and
wherein the first detectable probe comprises an oligonucleotide sequence selected from the group consisting of SEQ ID NOs:18-20, and the second detectable probe comprises an oligonucleotide sequence selected from the group consisting of SEQ ID NOs:21-22.

2. The method of claim 1, wherein the first primer comprises an oligonucleotide sequence selected from the group consisting of SEQ ID NOs:1-3, and the second primer comprises an oligonucleotide sequence selected from the group consisting of SEQ ID NOs: 4-6.

3. The method of claim 1, wherein:
each of said at least a first detectable probe and a second detectable probe are labeled with a donor fluorescent moiety and a corresponding acceptor moiety; and
step c) comprises detecting the presence or absence of fluorescence resonance energy transfer (FRET) between the donor fluorescent moiety and the acceptor moiety of the at least first and second detectable probes, wherein the presence or absence of fluorescence is indicative of the presence or absence of SARS-CoV-2 nucleic acids in the sample.

4. The method of claim 3, wherein the donor fluorescent moiety and the corresponding acceptor moiety on each of said first and second detectable probes are separated by 8-20 nucleotides, inclusive.

5. The method of claim 3, wherein each of said first and second detectable probes are labeled with a different donor fluorescent moiety selected from the group consisting of a fluorescein dye, a rhodamine dye, a cyanine dye, and a coumarin dye.

6. The method of claim 3, wherein said donor fluorescent moieties on said first and second detectable probes are the same, and are selected from the group consisting of Cy2, Cy3, Cy5, Cy 5.5 and Cy7.

7. The method of claim 1, wherein at least one of the primers and detectable probes includes a modified nucleotide.

8. The method of claim 7, wherein said modified nucleotide is selected from the group consisting of a t-butyl benzyl, a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolopyrimidine analog, a pseudo-dU, a nitro pyrrole, a nitro indole, 2′-0-methyl ribo-U, 2′-0-methyl ribo-C, an N4-ethyl-dC, and an N6-methyl-dA.

9. The method of claim 1, further comprising detecting a nucleic acid from one or more other viruses.

10. The method of claim 9, wherein the one or more other viruses is selected from the group consisting of influenza A, influenza B, influenza C, influenza D, respiratory syncytial virus (RSV), bat-coronavirus, severe acute respiratory syndrome (SARS) coronavirus (SARS-CoV), and Middle East respiratory syndrome (MERS) coronavirus (MERS-CoV).

11. The method of claim 2, wherein the first primer comprises a sequence of SEQ ID NO:3 and the second primer comprises a sequence of SEQ ID NO:4.

12. The method of claim 11, wherein the third primer comprises a sequence of SEQ ID NO:12 and the fourth primer comprises a sequence of SEQ ID NO:17.

13. The method of claim 12, wherein the second detectable probe comprises a sequence of SEQ ID NO:22.

14. The method of claim 1, further comprising a third set of primers, a fourth set of primers, a third detectable probe, and a fourth detectable probe;
wherein the third set of primers comprises a fifth primer having an oligonucleotide sequence selected from the group consisting of SEQ ID NO:23 and SEQ ID NOs: 7-9, and a sixth primer having an oligonucleotide sequence selected from the group consisting of SEQ ID NOs:24-25; and
wherein the fourth set of primers comprises a seventh primer having an oligonucleotide sequence consisting of SEQ ID NO:28 and an eighth primer having an oligonucleotide sequence consisting of SEQ ID NO:29; and
wherein the third detectable probe comprises an oligonucleotide having a sequence selected from the group consisting of SEQ ID NOs: 26-27, and the fourth detectable probe comprises an oligonucleotide having a sequence consisting of SEQ ID NO:30;
each of said third detectable probe and said fourth detectable probe being labeled with a different donor fluorescent moiety and a corresponding acceptor moiety; and
step c) further comprises detecting the presence or absence of fluorescence resonance energy transfer (FRET) between the donor fluorescent moiety and the acceptor moiety of the third and fourth detectable probes, wherein the presence or absence of fluorescence from the third detectable probe is indicative of the presence or absence of influenza A nucleic acids in the sample, and wherein the presence or absence of fluorescence from the fourth detectable probe is indicative of the presence or absence of influenza B nucleic acids in the sample.

15. The method of claim 14, wherein each of said third and fourth detectable probes are labeled with a different donor fluorescent moiety selected from the group consisting of a fluorescein dye, a rhodamine dye, a cyanine dye, and a coumarin dye.

16. The method of claim 14, wherein a donor fluorescent moiety is located on a terminal nucleotide of at least one of said first, second, third, and fourth detectable probes, and the corresponding acceptor moiety is located on the other terminal nucleotide of at least one of said first, second, third, and fourth detectable probes.

18. A kit for detecting SARS-CoV-2, comprising:
a first set of primers and a second set of primers;
a first detectable probe and a second detectable probe;
the first set of primers comprising a first primer having an oligonucleotide sequence selected from the group consisting of SEQ ID NOs:1-3 and SEQ ID NOs: 7-9, and a second primer having an oligonucleotide sequence selected from the group consisting of SEQ ID NOs:4-6 and SEQ ID NOs: 13-15;
the second set of primers comprising a third primer having an oligonucleotide sequence of SEQ ID NOs: 10-12, and a fourth primer having an oligonucleotide sequence selected from the group consisting of SEQ ID NOs:16-17; and
the first detectable probe comprising an oligonucleotide sequence of SEQ ID NOs:18-20; and
the second detectable probe comprising an oligonucleotide sequence selected from the group consisting of SEQ ID NOs:21-22.

19. The kit of claim 18, further comprising a third set of primers, a fourth set of primers, a third detectable probe, and a fourth detectable probe;
wherein the third set of primers comprises a fifth primer having an oligonucleotide sequence selected from the group consisting of SEQ ID NO:23 and SEQ ID NOs: 7-9, and a sixth primer having an oligonucleotide sequence selected from the group consisting of SEQ ID NOs:24-25; and
wherein the fourth set of primers comprises a seventh primer having an oligonucleotide sequence consisting of SEQ ID NO:28 and an eighth primer having an oligonucleotide sequence consisting of SEQ ID NO:29; and
wherein the third detectable probe comprises an oligonucleotide having a sequence selected from the group consisting of SEQ ID NOs: 26-27, and the fourth detectable probe comprises an oligonucleotide having a sequence consisting of SEQ ID NO:30.

20. A reaction vessel, comprising:
(a) a proximal end having an opening through which a sample is introducible;
(b) a distal end; and
(c) at least a first segment containing at least one nucleic acid extraction reagent, a second segment distal to the first segment and containing a wash reagent, and a third segment distal to the second segment and containing one or more amplification reagents, each of said segments being:
(i) defined by the tubule;
(ii) fluidly isolated, at least in part, by a fluid-tight seal formed by a bonding of opposed wall portions of the tubule to one another such that:
(A) the seal is broken by application of fluid pressure on a segment that is fluidly isolated in part by the seal; and
(B) the seal is capable of being clamped where the opposed wall portions of the tubule are bonded, without breaking the seal, to prevent the seal from being broken by application of fluid pressure on a segment that is fluidly isolated in part by the seal;
(iii) so expandable as to receive a volume of fluid expelled from another segment; and so compressible as to contain substantially no fluid when so compressed;
(d) a cap for closing the opening, the cap containing a chamber in fluid communication with the tubule, and the cap permitting free escape of gasses but retaining all liquid volumes and infectious agents in the tube;
(e) a rigid frame to which the tubules proximal and distal ends are held; and
(f) an integral tubule tensioning mechanism or an attachment of the tubule to the frame that pulls the tubule sufficiently taut so as to facilitate compression and flattening of the tubule;
(g) said reaction vessel containing
a first set of primers and a second set of primers;
a first detectable probe and a second detectable probe;
the first set of primers comprising a first primer having an oligonucleotide sequence selected from the group consisting of SEQ ID NOs:1-3 and SEQ ID NOs: 7-9, and a second primer having an oligonucleotide sequence selected from the group consisting of SEQ ID NOs:4-6 and SEQ ID NOs: 13-15;
the second set of primers comprising a third primer having an oligonucleotide sequence of SEQ ID NOs: 10-12, and a fourth primer having an oligonucleotide sequence selected from the group consisting of SEQ ID NOs:16-17; and
the first detectable probe comprising an oligonucleotide sequence of SEQ ID NOs:18-20; and
the second detectable probe comprising an oligonucleotide sequence selected from the group consisting of SEQ ID NOs:21-22.


SEQUENCE LISTING (30 sequences)

SEQ ID NO: 1 - DNA, 23 bp, Artificial sequence
  orf1ab forward primer
tgattgttacgatggtggctgta

SEQ ID NO: 2 - DNA, 23 bp, Artificial sequence
  orf1ab forward primer
gattgttacgatggtggctgtat

SEQ ID NO: 3 - DNA, 24 bp, Artificial sequence
  orf1ab forward primer
ggtggctgtattaatgctaaccaa

SEQ ID NO: 4 - DNA, 27 bp, Artificial sequence
  orf1ab reverse primer
agccttaccccatttattaaatggaaa

SEQ ID NO: 5 - DNA, 27 bp, Artificial sequence
  orf1ab reverse primer
ctagccttaccccatttattaaatgga

SEQ ID NO: 6 - DNA, 27 bp, Artificial sequence
  orf1ab reverse primer
tcataataaagtctagccttaccccat

SEQ ID NO: 7 - DNA, 19 bp, Artificial sequence
  orf1ab forward primer
cgtctgcggtatgtggaaa

SEQ ID NO: 8 - DNA, 21 bp, Artificial sequence
  orf1ab forward primer
ctgcggtatgtggaaaggtta

SEQ ID NO: 9 - DNA, 17 bp, Artificial sequence
  orf1ab forward primer
tcaactccgcgaaccca

SEQ ID NO: 10 - DNA, 28 bp, Artificial sequence
  N gene forward primer
cgaacaaactaaaatgtctgataatgga

SEQ ID NO: 11 - DNA, 21 bp, Artificial sequence
  N gene forward primer
ttacaaacattggccgcaaat

SEQ ID NO: 12 - DNA, 20 bp, Artificial sequence
  N gene forward primer
ggccgcaaattgcacaattt

SEQ ID NO: 13 - DNA, 22 bp, Artificial sequence
  orf1ab reverse primer
cgcaaacccgtttaaaaacgat

SEQ ID NO: 14 - DNA, 20 bp, Artificial sequence
  orf1ab reverse primer
caccgcaaacccgtttaaaa

SEQ ID NO: 15 - DNA, 18 bp, Artificial sequence
  orf1ab reverse primer
agacgggctgcacttaca

SEQ ID NO: 16 - DNA, 18 bp, Artificial sequence
  N gene reverse primer
tgagggtccaccaaacgt

SEQ ID NO: 17 - DNA, 19 bp, Artificial sequence
  N gene reverse primer
cgttcccgaaggtgtgact

SEQ ID NO: 18 - DNA, 32 bp, Artificial sequence
  orf1ab probe
  misc_feature: 5′ CY5.5
  misc_feature: 3′ BHQ-2
tcatcgtcaacaacctagacaaatcagctggt

SEQ ID NO: 19 - DNA, 28 bp, Artificial sequence
  orf1ab probe
  misc_feature: 5′ CY5.5
  misc_feature: 3′ phosphate
  misc_feature (12)..(13): BHQ-2
aacccatgcttcagtcagctgatgcaca

SEQ ID NO: 20 - DNA, 27 bp, Artificial sequence
  orf1ab probe
  misc_feature: 5′ CY5.5
  misc_feature: 3′ phosphate
  misc_feature (18)..(19): BHQ-2
tgcttcatcagctgatgcacaatcgtt

SEQ ID NO: 21 - DNA, 27 bp, Artificial sequence
  N gene probe
  misc_feature: 5′ CY5.5
  misc_feature: 3′ phosphate
  misc_feature (17)..(18): BHQ-2
caaaatcagcgaaatgcaccccgcatt

SEQ ID NO: 22 - DNA, 27 bp, Artificial sequence
  N gene probe
  misc_feature: 5′ CY5.5
  misc_feature: 3′ phosphate
  misc_feature (18)..(19): BHQ-2
agcgcttcagcgttcttcggaatgtcg

SEQ ID NO: 23 - DNA, 19 bp, Artificial sequence
  Influenza A forward primer
gctgcagtcctcgctcact

SEQ ID NO: 24 - DNA, 23 bp, Artificial sequence
  Influenza A reverse primer
ccaatcctgtcacctctgactaa

SEQ ID NO: 25 - DNA, 23 bp, Artificial sequence
  Influenza A reverse primer
  misc_feature: R=A or G
  misc_feature: Y=C or T
ccaatcctgtcacctctracyaa

SEQ ID NO: 26 - DNA, 22 bp, Artificial sequence
  Influenza A probe
  misc_feature: 5′ FAM
  misc_feature: 3′ BHQ-1
cggtgagcgtgaacacaaatcc

SEQ ID NO: 27 - DNA, 22 bp, Artificial sequence
  Influenza A probe
  misc_feature: 5′ FAM
  misc_feature: 3′ BHQ-1
cggtgagcgtgaawacaaaccc

SEQ ID NO: 28 - DNA, 21 bp, Artificial sequence
  Influenza B forward primer
caagggctctttgccatgaaa

SEQ ID NO: 29 - DNA, 15 bp, Artificial sequence
  Influenza B reverse primer
gggtccgggagcaac

SEQ ID NO: 30 - DNA, 24 bp, Artificial sequence
  Influenza B probe
  misc_feature: 5′ Cal Fluor Red 610
  misc_feature: 3′ BHQ-2
aattcctgcttcaaagtttatggt